(19) United States
(12) Patent Application Publication
(10) Pub. No.: US 2023/0355962 A1
(43) Pub. Date: Nov. 9, 2023

(54) ADVANCED SURGICALLY IMPLANTABLE TECHNOLOGIES

(71) Applicant: COCHLEAR LIMITED, Macquarie University, NSW (AU)

(72) Inventors: Martin Joseph SVEHLA, Macquarie University (AU); Oliver John RIDLER, Macquarie University (AU); Charles Roger Aaron LEIGH, Macquarie University (AU); Milind Chandrakant RAJE, Macquarie University (AU); Daanish FAIZ, Macquarie University (AU)

(21) Appl. No.: 18/029,500

(22) PCT Filed: Sep. 30, 2021
(86) PCT No.: PCT/IB2021/058997

§ 371 Date: Mar. 30, 2023

Related U.S. Application Data
(60) Provisional application No. 63/085,676, filed on Sep. 30, 2020.

(51) Int. Cl. A61N 1/378; A61N 1/05; A61N 1/372; A61N 1/375

(52) U.S. Cl. CPC A61N 1/0541; A61N 1/37229; A61N 1/375; A61N 1/3787

(57) ABSTRACT

An apparatus, including an implantable housing, implantable electronics hermetically sealed within at least a portion of the implantable housing and an implantable inductance coil, wherein an outer diameter of the inductance coil is less than 17.5 mm and located in its entirety within 5 mm of portion(s) of the housing, and wherein the apparatus is fully implantable within a human

### CROSS-REFERENCE TO RELATED APPLICATIONS

This application claims priority to U.S. Provisional Application No. 63/085,676, entitled ADVANCED SURGICALLY IMPLANTABLE TECHNOLOGIES, filed on Sep. 30, 2020, naming Martin Joseph SVEHLA of Macquarie University, Australia as an inventor, the entire contents of that application being incorporated herein by reference in its entirety.

### BACKGROUND

Medical devices having one or more implantable components, generally referred to herein as implantable medical devices, have provided a wide range of therapeutic benefits to recipients over recent decades. In particular, partially or fully-implantable medical devices such as hearing prostheses (e.g., bone conduction devices, mechanical stimulators, cochlear implants, etc.), implantable pacemakers, defibrillators, functional electrical stimulation devices, and other implantable medical devices, have been successful in performing lifesaving and/or lifestyle enhancement functions and/or recipient monitoring for a number of years.

The types of implantable medical devices and the ranges of functions performed thereby have increased over the years. For example, many implantable medical devices now often include one or more instruments, apparatus, sensors, processors, controllers or other functional mechanical or electrical components that are permanently or temporarily implanted in a recipient. These functional devices are typically used to diagnose, prevent, monitor, treat, or manage a disease/injury or symptom thereof, or to investigate, replace or modify the anatomy or a physiological process. Many of these functional devices utilize power and/or data received from external devices that are part of, or operate in conjunction with, the implantable medical device.

### SUMMARY

In an exemplary embodiment, there is an apparatus, comprising an implantable housing, implantable electronics hermetically sealed within at least a portion of the implantable housing, and, an implantable inductance coil, wherein an outer diameter of the inductance coil is less than 17.5 mm and located in its entirety within 5 mm of portion(s) of the housing, and wherein the apparatus is fully implantable within a human.

In an exemplary embodiment, there is a system, comprising an implantable component configured to be fully implantable in a human, and an external component configured to be, in its totality, retained on a head of the human via at least one of behind-the-ear structure or in-the-ear structure, wherein the external component is configure to be in inductance signal communication with the implantable component.

In an exemplary embodiment, there is a surgical implantation method, comprising accessing a location inside a human surgically implanting an implantable portion of a hearing prosthesis at the location, wherein the hearing prosthesis is one of a middle ear implant or a cochlear implant powered by transcutaneous inductance power transfer, and at least one of the action of accessing the location inside the human is executed by incising skin with lengths greater than ½ inch only within 3 inches of an inner surface of an ear canal of the human or the implantable portion has a height exceeding at least half a maximum width, and the action of accessing the location is executed by drilling and/or excavating into bone only within 2 inches of an inner surface of the ear canal of the human.

In an exemplary embodiment, there is an apparatus, comprising a housing made of a titanium alloy or a glass material, the housing being a biocompatible housing suitable for implantation in a human recipient, implantable electronics hermetically sealed within at least a portion of the housing, the implantable electronics comprising circuitry configured to receive a signal from an implantable inductance coil and output a stimulation signal based on the received signal to stimulate tissue of the recipient, and the implantable inductance coil, wherein an outer diameter of the inductance coil is less than 17.5 mm and located in its entirety within 5 mm of portion(s) of the housing, and wherein the apparatus is fully implantable within the human.

### BRIEF DESCRIPTION OF THE DRAWINGS

Embodiments are described below with reference to the attached drawings, in which:

FIG. 1A is a perspective view of an exemplary hearing prosthesis in which at least some of the teachings detailed herein are applicable;

FIGS. 1B-1D are quasi functional diagrams of an exemplary device to which some embodiments may be applicable;

FIGS. 1E and 2A and 2B and 2C and 1F present some schematics related to base technologies associated with some embodiments;

FIGS. 3-10 depict various schematics of various embodiments of the teachings detailed herein;

FIGS. 11-17 and 20 and 25 and 27 depict various implantation regimes according to some exemplary embodiments;

FIGS. 18 and 19 and 21 and 22 and 26 present some additional various embodiments of the teachings detailed herein; and

FIG. 23 presents an exemplary flowchart for an exemplary method.

### DETAILED DESCRIPTION

Merely for ease of description, the techniques presented herein are primarily described herein with reference to an illustrative medical device, namely a hearing prosthesis. First introduced is a cochlear implant. The techniques presented herein may also be used with a variety of other medical devices that, while providing a wide range of therapeutic benefits to recipients, patients, or other users, may benefit from the teachings herein used in other medical devices. For example, any techniques presented herein described for one type of hearing prosthesis, such as a cochlear implant, corresponds to a disclosure of another embodiment of using such teaching with another hearing prosthesis, including bone conduction devices (percutaneous, active transcutaneous and/or passive transcutaneous), middle ear auditory prostheses, direct acoustic stimulators, and also utilizing such with other electrically simulating auditory prostheses (e.g., auditory brain stimulators), etc. The techniques presented herein can be used with implantable / implanted microphones, whether or not used as part of a hearing prosthesis (e.g., a body noise or other monitor, whether or not it is part of a hearing prosthesis) and/or external microphones. The techniques presented herein can also be used with vestibular devices (e.g., vestibular implants), sensors, seizure devices (e.g., devices for monitoring and/or treating epileptic events, where applicable), sleep apnea devices, electroporation, etc., and thus any disclosure herein is a disclosure of utilizing such devices with the teachings herein, providing that the art enables such. The teachings herein can also be used with conventional hearing devices, such as telephones and ear bud devices connected MP3 players or smart phones or other types of devices that can provide audio signal output. Indeed, the teachings herein can be used with specialized communication devices, such as military communication devices, factory floor communication devices, professional sports communication devices, etc.

By way of example, any of the technologies detailed herein which are associated with components that are implanted in a recipient can be combined with information delivery technologies disclosed herein, such as for example, devices that evoke a hearing percept, to convey information to the recipient. By way of example only and not by way of limitation, a sleep apnea implanted device can be combined with a device that can evoke a hearing percept so as to provide information to a recipient, such as status information, etc. In this regard, the various sensors detailed herein and the various output devices detailed herein can be combined with such a non-sensory prosthesis or any other nonsensory prosthesis that includes implantable components so as to enable a user interface, as will be described herein, that enables information to be conveyed to the recipient, which information is associated with the implant.

While the teachings detailed herein will be described for the most part with respect to hearing prostheses, in keeping with the above, it is noted that any disclosure herein with respect to a hearing prosthesis corresponds to a disclosure of another embodiment of utilizing the associated teachings with respect to any of the other prostheses noted herein, whether a species of a hearing prosthesis, or a species of a sensory prosthesis.

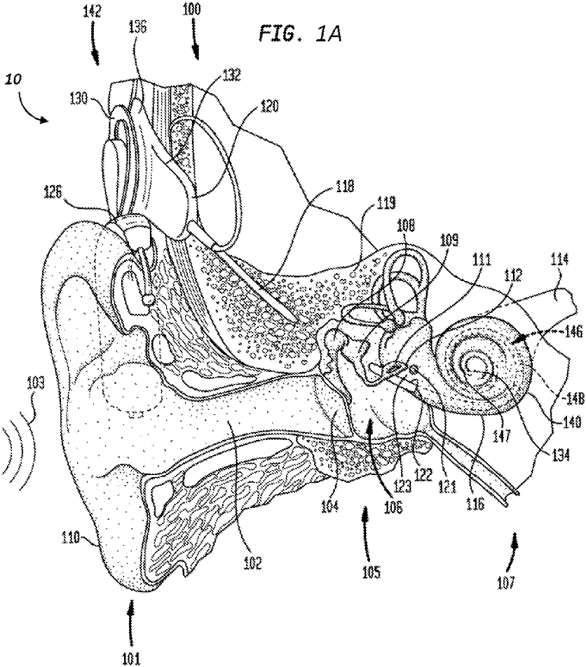

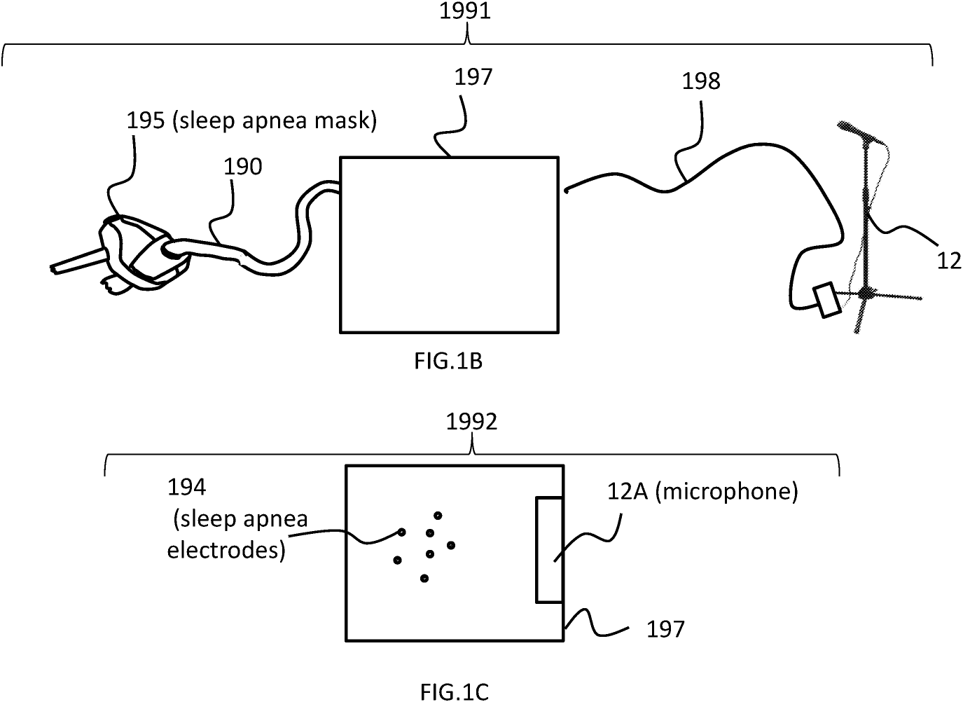

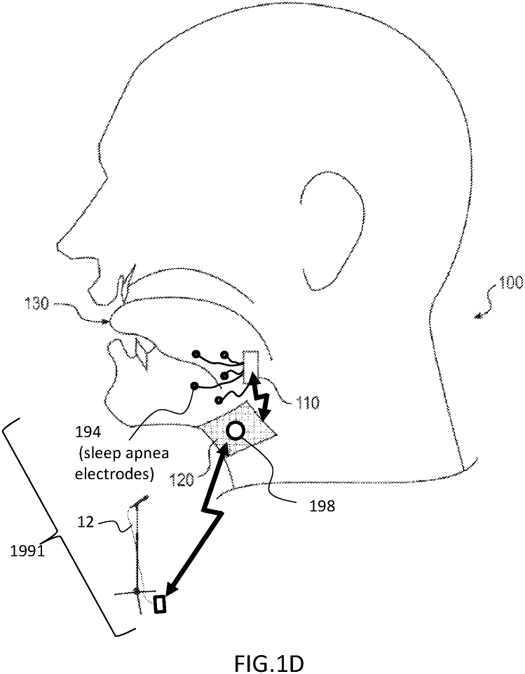

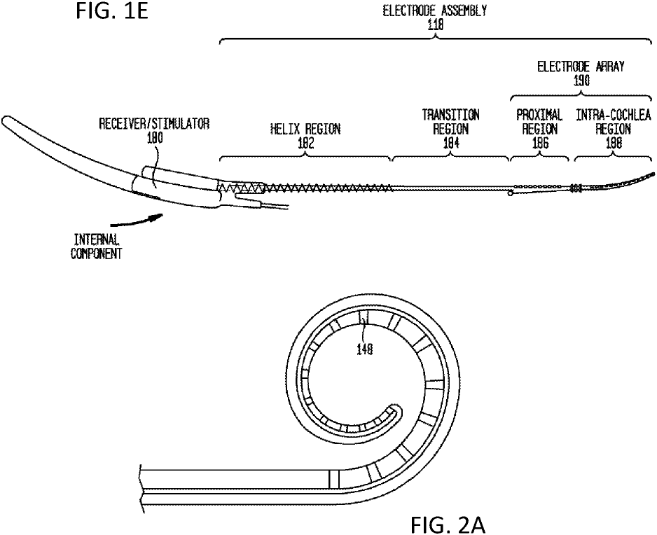

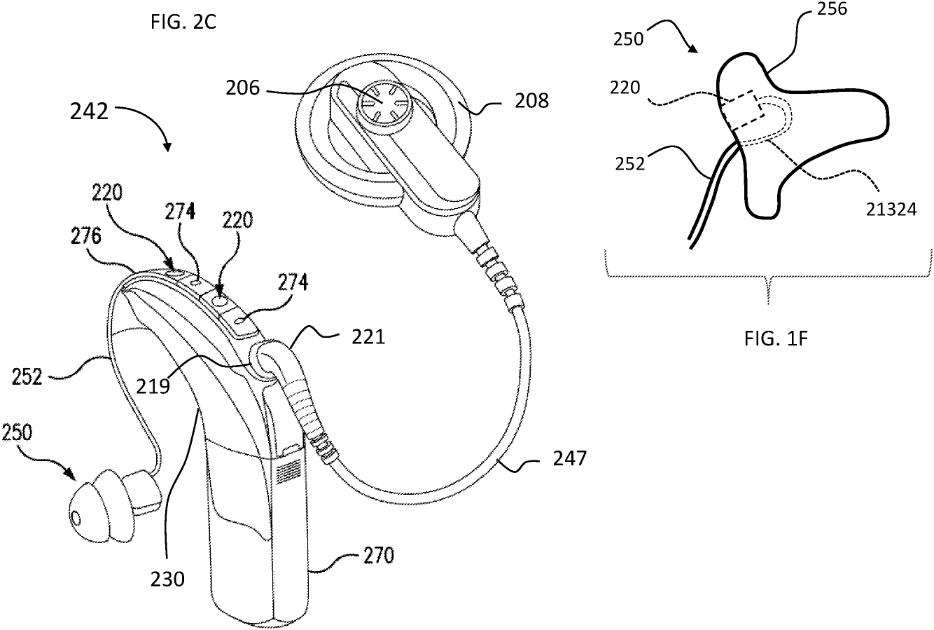

FIG. 1A is perspective view of a partially implantable cochlear implant, referred to as cochlear implant 100, implanted in a recipient. The cochlear implant 100 is part of a system 10 that can include external component(s), as will be detailed below.

The recipient has an outer ear 101, a middle ear 105, and an inner ear 107. Components of outer ear 101, middle ear 105, and inner ear 107 are described below, followed by a description of cochlear implant 100.

In a fully functional ear, outer ear 101 comprises an auricle 110 and an ear canal 102. An acoustic pressure or sound wave 103 is collected by auricle 110 and channeled into and through ear canal 102. Disposed across the distal end of ear canal 102 is a tympanic membrane 104 which vibrates in response to sound wave 103. This vibration is coupled to oval window or fenestra ovalis 112 through three bones of middle ear 105, collectively referred to as the ossicles 106 and comprising the malleus 108, the incus 109, and the stapes 111. Bones 108, 109, and 111 of middle ear 105 serve to filter and amplify sound wave 103, causing oval window 112 to articulate, or vibrate in response to vibration of tympanic membrane 104. This vibration sets up waves of fluid motion of the perilymph within cochlea 140. Such fluid motion, in turn, activates tiny hair cells (not shown) inside of cochlea 140. Activation of the hair cells causes appropriate nerve impulses to be generated and transferred through the spiral ganglion cells (not shown) and auditory nerve 114 to the brain (also not shown) where they are perceived as sound.

As shown, cochlear implant 100 comprises one or more components which are temporarily or permanently implanted in the recipient. Cochlear implant 100 is shown in FIG. 1A with an external device 142, that is part of system 10 (along with cochlear implant 100), which, as described below, is configured to provide power to the cochlear implant.

In the illustrative arrangement of FIG. 1A, external device 142 may comprise a power source (not shown) disposed in a Behind-The-Ear (BTE) unit 126. External device 142 also includes components of a transcutaneous energy transfer link, referred to as an external energy transfer assembly. The transcutaneous energy transfer link is used to transfer power and/or data to cochlear implant 100. Various types of energy transfer, such as infrared (IR), electromagnetic, capacitive and inductive transfer, may be used to transfer the power and/or data from external device 142 to cochlear implant 100. In the illustrative embodiments of FIG. 1A, the external energy transfer assembly comprises an external coil 130 that forms part of an inductive radio communication link. External coil 130 is typically a wire antenna coil comprised of multiple turns of electrically insulated single-strand or multi-strand platinum or gold wire. External device 142 also includes a magnet (not shown) positioned within the turns of wire of external coil 130. It should be appreciated that the external device shown in FIG. 1A is merely illustrative, and other external devices may be used with embodiments of the present invention.

Cochlear implant 100 comprises an internal energy transfer assembly 132 which may be positioned in a recess of the temporal bone adjacent auricle 110 of the recipient. As detailed below, internal energy transfer assembly 132 is a component of the transcutaneous energy transfer link and receives power and/or data from external device 142. In the illustrative embodiment, the energy transfer link comprises an inductive RF link, and internal energy transfer assembly 132 comprises a primary internal coil 136. Internal coil 136 is typically a wire antenna coil comprised of multiple turns of electrically insulated single-strand or multi-strand platinum or gold wire.

Cochlear implant 100 further comprises a main implantable component 120 and an elongate stimulating assembly 118. In embodiments of the present invention, internal energy transfer assembly 132 and main implantable component 120 are hermetically sealed within a biocompatible housing. In embodiments of the present invention, main implantable component 120 includes a sound processing unit (not shown) to convert the sound signals received by the implantable microphone in internal energy transfer assembly 132 to data signals. Main implantable component 120 further includes a stimulator unit (also not shown) which generates electrical stimulation signals based on the data signals. The electrical stimulation signals are delivered to the recipient via elongate stimulating assembly 118.

Elongate stimulating assembly 118 has a proximal end connected to main implantable component 120, and a distal end implanted in cochlea 140. Stimulating assembly 118 extends from main implantable component 120 to cochlea 140 through mastoid bone 119. In some embodiments stimulating assembly 118 may be implanted at least in basal region 116, and sometimes further. For example, stimulating assembly 118 may extend towards apical end of cochlea 140, referred to as cochlea apex 134. In certain circumstances, stimulating assembly 118 may be inserted into cochlea 140 via a cochleostomy 122. In other circumstances, a cochleostomy may be formed through round window 121, oval window 112, the promontory 123 or through an apical turn 147 of cochlea 140.

Stimulating assembly 118 comprises a longitudinally aligned and distally extending array 146 of electrodes 148, disposed along a length thereof. As noted, a stimulator unit generates stimulation signals which are applied by stimulating contacts 148, which, in an exemplary embodiment, are electrodes, to cochlea 140, thereby stimulating auditory nerve 114. In an exemplary embodiment, stimulation contacts can be any type of component that stimulates the cochlea (e.g., mechanical components, such as piezoelectric devices that move or vibrate, thus stimulating the cochlea (e.g., by inducing movement of the fluid in the cochlea), electrodes that apply current to the cochlea, etc.). Embodiments detailed herein will generally be described in terms of an electrode assembly 118 utilizing electrodes as elements 148. It is noted that alternate embodiments can utilize other types of stimulating devices. Any device, system, or method of stimulating the cochlea via a device that is located in the cochlea can be utilized in at least some embodiments. In this regard, any implantable array that stimulates tissue, such as a retinal implant array, or a spinal array, or a pacemaker array, etc., is encompassed within the teachings herein unless otherwise noted.

As noted, cochlear implant 100 comprises a partially implantable prosthesis, as contrasted to a totally implantable prosthesis that is capable of operating, at least for a period of time, without the need for external device 142. Therefore, cochlear implant 100 does not comprise a rechargeable power source that stores power received from external device 142, as contrasted to an embodiment where there is an implantable rechargeable power source (e.g., a rechargeable battery). During operation of cochlear implant 100, the power is transferred from the external component to the implanted component via the link, and distributed to the various other implanted components as needed.

It is noted that the teachings detailed herein and/or variations thereof can be utilized with a totally implantable prosthesis. That is, in an alternate embodiment of the cochlear implants or other hearing prostheses detailed herein, the prostheses are totally implantable prostheses, such as where there is an implanted microphone and sound processor and battery.

FIG. 1B provides a schematic of an exemplary conceptual sleep apnea system 1991. Here, this exemplary sleep apnea system utilizes a microphone 12 (represented conceptually) to capture a person’s breathing or otherwise the sounds made by a person while sleeping. The microphone transduces the captured sound into an electrical signal which is provided via electrical leads 198 to the main unit 197, which includes a processor unit that can evaluate the signal from leads 198 or, in another arrangement, unit 197 is configured to provide that signal to a remote processing location via the Internet or the like, where the signal was evaluated. Upon an evaluation that an action should be taken or otherwise can be utilitarian taken by the sleep apnea system 1991, the unit 197 activates to implement sleep apnea countermeasures, which countermeasures are conducted by a hose 1902 sleep apnea mask 195. By way of example only and not by way of limitation, pressure variations can be used to treat the sleep apnea upon an indication of such an occurrence.

In an exemplary embodiment, the advanced implantation methods and devices detailed herein can be utilized to treat sleep apnea. Specifically, the electrodes of the implant disclosed below can be utilized in place of the electrodes 194 (placed accordingly, of course), and the implant can be of a configuration to treat sleep apnea. In this regard, in an exemplary embodiment, the implantable components detailed herein can be located at locations to treat sleep apnea in accordance with the teachings herein, with the requisite modification if necessary or otherwise utilitarian to implement such.

FIGS. 1C and 1D provide another exemplary schematic of another exemplary conceptual sleep apnea system 1992. Here, the sleep apnea system is different from that of FIG. 1B in that electrodes 194 (which can be implanted in some embodiments) are utilized to provide stimulation to the human who is experiencing a sleep apnea scenario. FIG. 1C illustrates an external unit, and FIG. 1D illustrates the external unit 120 and an implanted unit 110 in signal communication via an inductance coil 707 of the external unit and a corresponding implanted inductance coil (not shown) of the implanted unit, according to which the teachings herein can be applicable. Implanted unit 110, can be configured for implantation in a recipient, in a location that permits it to modulate nerves of the recipient 100 via electrodes 194. In treating sleep apnea, implant unit 110 and/or the electrodes thereof can be located on a genioglossus muscle of a patient. Such a location is suitable for modulation of the hypoglossal nerve, branches of which run inside the genioglossus muscle.

External unit 120 can be configured for location external to a patient, either directly contacting, or close to the skin of the recipient. External unit 120 may be configured to be affixed to the patient, for example, by adhering to the skin of the patient, or through a band or other device configured to hold external unit 120 in place. Adherence to the skin of external unit 120 may occur such that it is in the vicinity of the location of implant unit 110 so that, for example, the external unit 120 can be in signal communication with the implant unit 110 as conceptually shown, which communication can be via an inductive link or an RF link or any link that can enable treatment of sleep apnea using the implant unit and the external unit. External unit 120 can include a processor unit 198 that is configured to control the stimulation executed by the implant unit 110. In this regard, processor unit 198 can be in signal communication with microphone 12, via electrical leads, such as in an arrangement where the external unit 120 is a modularized component, or via a wireless system, such as conceptually represented in FIG. 1D.

A common feature of both of these sleep apnea treatment systems is the utilization of the microphone to capture sound, and the utilization of that captured sound to implement one or more features of the sleep apnea system. In some embodiments, the teachings herein are used with the sleep apnea device just detailed.

Returning back to hearing prosthesis devices, and in particular a cochlear implant,

FIG. 1E is a side view of the internal component of cochlear implant 100 without the other components of system 10 (e.g., the external components). Cochlear implant 100 comprises a receiver/stimulator 180 (combination of main implantable component 120 and internal energy transfer assembly 132) and a stimulating assembly or lead 118. Stimulating assembly 118 includes a helix region 182, a transition region 184, a proximal region 186, and an intra-cochlear region 188. Proximal region 186 and intra-cochlear region 188 form an electrode array assembly 190. In an exemplary embodiment, proximal region 186 is located in the middle-ear cavity of the recipient after implantation of the intra-cochlear region 188 into the cochlea. Thus, proximal region 186 corresponds to a middle-ear cavity sub-section of the electrode array assembly 190. Electrode array assembly 190, and in particular, intra-cochlear region 188 of electrode array assembly 190, supports a plurality of electrode contacts 148. These electrode contacts 148 are each connected to a respective conductive pathway, such as wires, PCB traces, etc. (not shown) which are connected through lead 118 to receiver/stimulator 180, through which respective stimulating electrical signals for each electrode contact 148 travel.

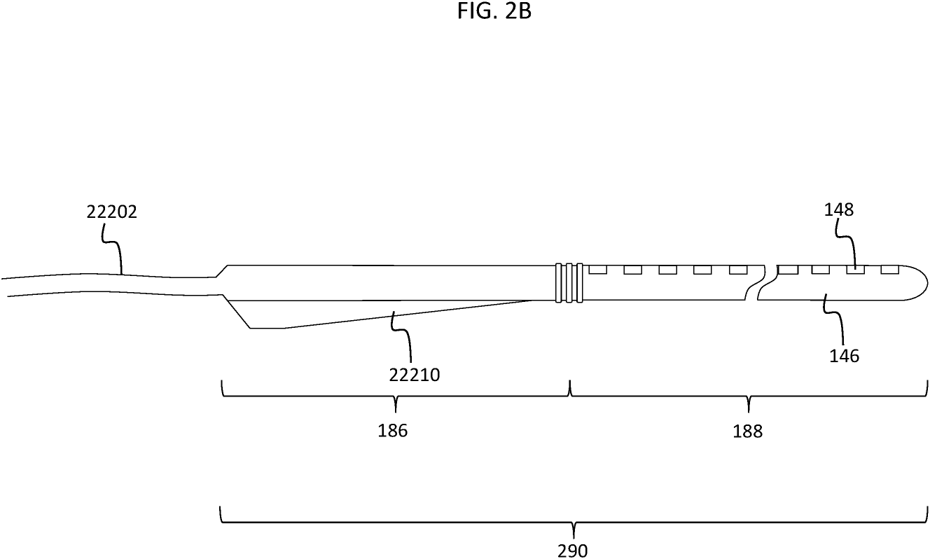

FIG. 2A is a side view of electrode array assembly 190 in a curled orientation, as it would be when inserted in a recipient’s cochlea, with electrode contacts 148 located on the inside of the curve. FIG. 2A depicts the electrode array of FIG. 1B in situ in a patient’s cochlea 140.

FIG. 2B depicts a side view of a device 290 corresponding to a cochlear implant electrode array assembly that can include some or all of the features of electrode array assembly 190 of FIG. 1B. More specifically, in an exemplary embodiment, stimulating assembly 118 includes electrode array assembly 290 instead of electrode array assembly 190 (i.e., 190 is replaced with 290).

Electrode array assembly 290 includes a cochlear implant electrode array componentry of the 190 assembly above. Note also element 22210, which is a quasi-handle like device utilized with utilitarian value vis-à-vis inserting the 188 section into a cochlea. By way of example only and not by way of limitation, element 22210, which is a silicone body that extends laterally away from the longitudinal axis of the electrode array assembly 290, and has a thickness that is less than that of the main body of the assembly (the portion through which the electrical leads that extend to the electrodes extend to the elongate lead assembly 22202). The thickness combined with the material structure is sufficient so that the handle can be gripped at least by a tweezers or the like during implantation and by application of a force on to the tweezers, the force can be transferred into the electrode array assembly 290 so that section 188 can be inserted into the cochlea.

FIG. 2C presents additional details of an external component assembly 242, corresponding to external component 142 above.

External assembly 242 typically comprises a sound transducer 220 for detecting sound, and for generating an electrical audio signal, typically an analog audio signal. In this illustrative arrangement, sound transducer 220 is a microphone. In alternative arrangements, sound transducer 220 can be any device now or later developed that can detect sound and generate electrical signals representative of such sound. An exemplary alternate location of sound transducer 220 will be detailed below. As will be detailed below, a sound transducer can also be located in an ear piece, which can utilize the “funneling” features of the pinna for more natural sound capture (more on this below).

External assembly 242 also comprises a signal processing unit, a power source (not shown), and an external transmitter unit. External transmitter unit 206 (sometimes herein referred to as a headpiece) comprises an external coil 208 and, a magnet (not shown) secured directly or indirectly to the external coil 208. The signal processing unit processes the output of microphone 220 that is positioned, in the depicted arrangement, by outer ear 201 of the recipient. The signal processing unit generates coded signals using a signal processing apparatus (sometimes referred to herein as a sound processing apparatus), which can be circuitry (often a chip) configured to process received signals - because element 230 contains this circuitry, the entire component 230 is often called a sound processing unit or a signal processing unit. These coded signals can be referred to herein as a stimulation data signals, which are provided to external transmitter unit 206 via a cable 247. In this exemplary arrangement of FIG. 1D, cable 247 includes connector jack 221 which is bayonet fitted into receptacle 219 of the signal processing unit 230 (an opening is present in the dorsal spine, which receives the bayonet connector, in which includes electrical contacts to place the external transmitter unit into signal communication with the signal processor 230). It is also noted that in alternative arrangements, the external transmitter unit is hardwired to the signal processor subassembly 230. That is, cable 247 is in signal communication via hardwiring, with the signal processor subassembly. (The device of course could be disassembled, but that is different than the arrangement shown in FIG. 1D that utilizes the bayonet connector.) Conversely, in some embodiments, there is no cable 247. Instead, there is a wireless transmitter and/or transceiver in the housing of component 230 and/or attached to the housing (e.g., a transmitter / transceiver can be attached to the receptacle 219) and the headpiece can include a receiver and/or transceiver, and can be in signal communication with the transmitter / transceiver of / associated with element 230.

FIG. 1F provides additional details of an exemplary in-the-ear (ITE) component 250. The overall component containing the signal processing unit is, in this illustration, constructed and arranged so that it can fit behind outer ear 201 in a BTE (behind-the-ear) configuration, but may also be worn on different parts of the recipient’s body or clothing.

In some arrangements, the signal processor (also referred to as the sound processor) may produce electrical stimulations alone, without generation of any acoustic stimulation beyond those that naturally enter the ear. While in still further arrangements, two signal processors may be used. One signal processor is used for generating electrical stimulations in conjunction with a second speech processor used for producing acoustic stimulations.

As shown in FIG. 1F, an ITE component 250 is connected to the spine of the BTE (a general term used to describe the part to which the battery 270 attaches, which contains the signal (sound) processor and supports various components, such as the microphone - more on this below) through cable 252 (and thus connected to the sound processor / signal processor thereby). ITE component 250 includes a housing 256, which can be a molding shaped to the recipient. Inside ITE component 250 there is provided a sound transducer 220 that can be located on element 250 so that the natural wonders of the human ear can be utilized to funnel sound in a more natural manner to the sound transducer of the external component. In an exemplary arrangement, sound transducer 242 is in signal communication with remainder of the BTE unit via cable 252, as is schematically depicted in FIG. 1F via the sub cable extending from sound transducer 242 to cable 252. Shown in dashed lines are leads 21324 that extend from transducer 220 to cable 252. Not shown is an air vent that extends from the left side of the housing 256 to the right side of the housing (at or near the tip on the right side) to balance air pressure “behind” the housing 256 and the ambient atmosphere when the housing 256 is in an ear canal.

Also, FIG. 1D shows a removable power component 270 (sometimes battery back, or battery for short) directly attached to the base of the body / spine 230 of the BTE device. As seen, the BTE device in some embodiments includes control buttons 274. The BTE device may have an indicator light 276 on the earhook to indicate operational status of signal processor. Examples of status indications include a flicker when receiving incoming sounds, low rate flashing when power source is low or high rate flashing for other problems.

In one arrangement, external coil 130 transmits electrical signals to the internal coil via an inductance communication link. The internal coil is typically a wire antenna coil comprised of at least one, or two or three or more turns of electrically insulated single-strand or multi-strand platinum or gold wire. The electrical insulation of the internal coil is provided by a flexible silicone molding (not shown). In use, internal receiver unit may be positioned in a recess of the temporal bone adjacent to outer ear 101 of the recipient.

With the above as a primer (the above should be considered base technologies from which we build upon, and are not part of the invention, but the teachings below can use any one or more of these features in some embodiments, providing that the art enables such), embodiments are directed to cochlear implants and middle ear implants and DACS that, in some embodiments, utilize one or more of the teachings above, albeit modified in at least some instances, to practice the teachings herein.

Also, while the teachings associated above are typically directed towards a cochlear implant, the disclosure of such and any teachings herein relating to such also correspond to a disclosure of an implantable / implanted device that is a middle ear implant or a DACS, that utilizes some of the pertinent teachings (e.g., both will utilize the inductance communication for power, for example). The output will be different (mechanical stimulation vs. electricity), and thus the “stimulator” features will also be different, as is understood in the art.

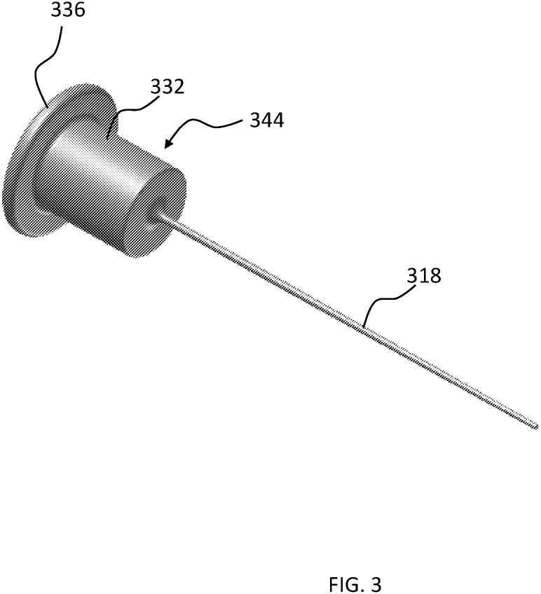

FIG. 3 presents a portion of an exemplary implantable component 344 in an exemplary embodiment of a cochlear implant (it is a portion because the electrode array and the associated lead assembly is not shown - more on this below - briefly, a component corresponding to that shown in FIG. 2B extends from the end of element 318, for example, elongate lead assembly 22202 extends from the end of element 318, and the electrode array 290 is attached thereto so that it can be inserted into a cochlea - note that 318 is not shown to scale - it could be much shorter (or longer) - and also, the elongate lead assembly could be located at the position where the end of element 318 is located). More specifically, in some exemplary embodiments, what is shown in FIG. 3 is a monolithic component (one piece -by analogy, an Egyptian obelisk carved from a single piece of rock is monolithic - the Washington Monument, while an obelisk, is made of multiple rocks mortared together - that is not monolithic - it is an integral body (the Egyptian obelisk is also an integral body)). It can be made from a single piece of material, such as for example, a polymer. In some other embodiments, what is shown in FIG. 3 is an integral body - the portions visible can be made of a single piece of titanium, for example, and a “lid” can be made of a ceramic or some other material - more on this in a moment. What is seen in FIG. 3 is a housing that houses the electronic components of an implantable portion of a cochlear implant (e.g., the inductance coil, the receiver-stimulator unit, and the electrical leads to the electrode array). It is noted that in an exemplary embodiment, one or more or all of the portion shown in FIG. 3 can be covered by a coating of silicone.

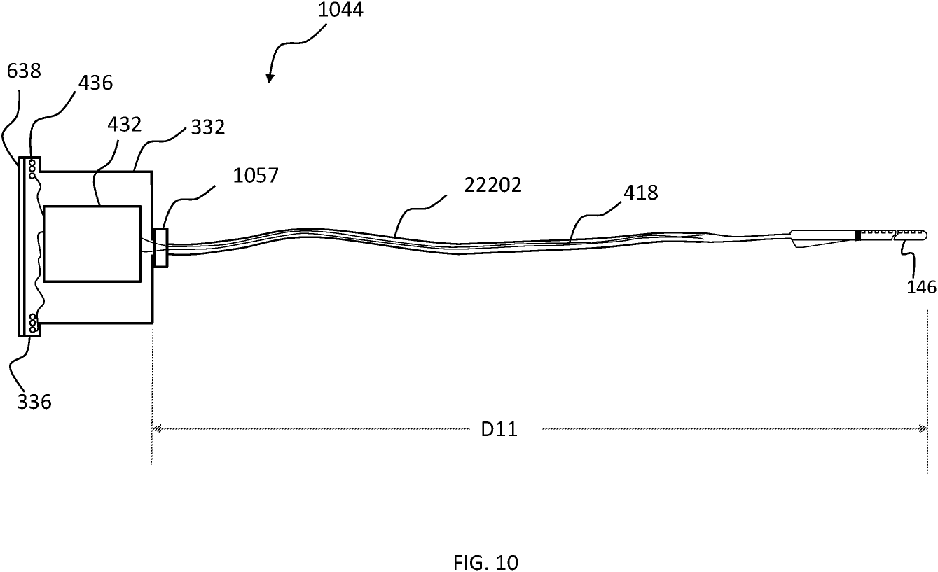

Implantable component 344, or more accurately, the housing of the implantable component 344, includes a housing portion 336 that houses an inductance coil and a housing portion 332 that houses the receiver-stimulator. The housing of the implantable component 344 further includes housing portion 318, which houses the electrical leads that extend from the receiver-stimulator to the electrode array (not shown). It is briefly noted that in some embodiments, the housing portion 318 is instead a lead assembly that is flexible and corresponds to the lead assembly of the helix region and/or the transition region of the embodiment of FIG. 2B above (the part that extends from the receiver-stimulator 180 to the electrode array 190). In some embodiments, the portions to the right of housing portion 332 corresponds to the embodiment of FIG. 2B (element 318 can correspond to element 22202 of FIG. 2B — see FIG. 10 and the description thereof below as an example). Additional details of this will be described below. For the moment, embodiments will be described where the part that extends from the housing portion 332 is another housing portion as opposed to a lead assembly. It is noted that any embodiment disclosing the former corresponds to the disclosure of an alternate embodiment disclosing the latter.

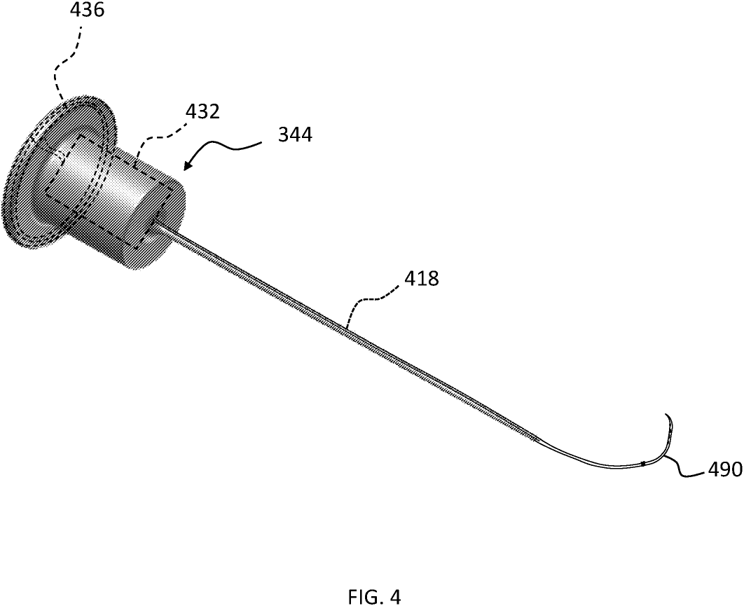

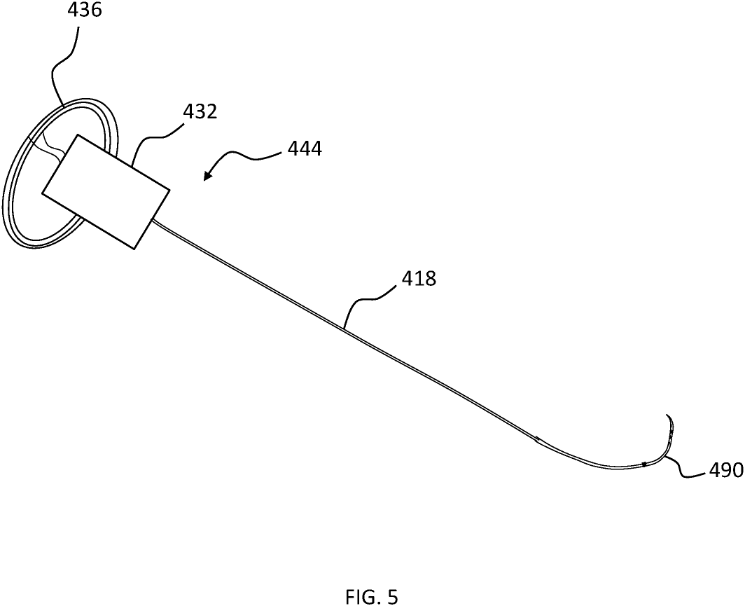

FIG. 4 presents these various components located within the various portions of the housing, and FIG. 5 shows the components without the housing. As can be seen, there is inductance coil 436 connected by leads to receiver-stimulator 432, which is connected to the electrode array 490 via leads 418. FIG. 4 thus shows the various elements of the implantable portion 344 of this exemplary cochlear implant (again, silicone might be coated over the components shown). In an exemplary embodiment, a ferrite core is located in-plane with the coil / the coil extends about a ferrite core, and that core can be in the housing portion 344.

The coil 436 can be a modification of the inductance coils detailed above and/or variations thereof and/or variations of known inductance coils utilized in the art. In this regard, the inductance coil can be made of a wire of platinum or gold or copper or otherwise any appropriate material that can enable the teachings detailed herein. In these embodiments, the inductance coil 436 has an overall geometry that is smaller than that which is traditionally utilized, such as those detailed above with respect to FIG. 1A. The receiver-stimulator 432 can include circuitry or otherwise correspond to an electronics circuit (it can be in the form of a PCB, for example, or a chip, etc.) that is configured to have the functionality of a receiver-stimulator of a cochlear implant (or a middle ear implant or a DACS, in other embodiments — more on this below). In an exemplary embodiment, this can correspond to a miniaturized version of commercially available receiver-stimulators of a cochlear implant (or a middle ear implant or a DACS). In an exemplary embodiment, the function of the receiver-stimulator 432, is to receive an alternating current from the coil 436 (induced via the external component), and generate output signals based on that alternating current which are applied to the various electrodes of the electrode array 490 (in some embodiments, there are 22 electrodes on the array 490 (and a “ground” or common electrode on the housing portion 318 or 332 or elsewhere — in some embodiments, the housing serves as a ground electrode) with a specific timing between the electrodes. This application of output to the electrodes corresponds to the output to electrodes of a traditional cochlear implant, and the functionality thereof corresponds to the functionality of a traditional cochlear implant.

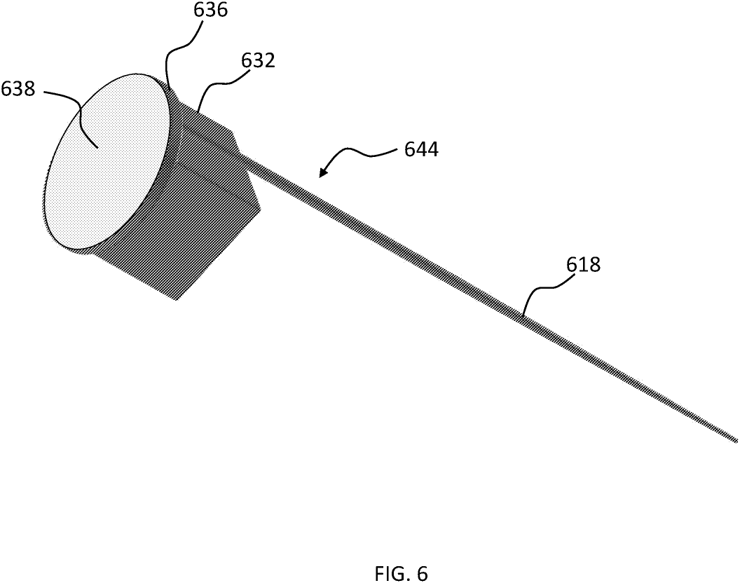

FIG. 6 presents another exemplary embodiment of a portion of an implantable component 644 of a cochlear implant according to an exemplary embodiment. Here, there is a housing portion 618 housing the leads 418, a housing portion 632 housing the receiver-stimulator, and a housing portion 636 housing the inductance coil. Further, there is a lid 638 that covers the housing portion 636. In this exemplary embodiment, the lid 638 is a ceramic component that is relatively transparent to inductance signals transmitted from the external component. In an exemplary embodiment, this lid arrangement exists with respect to the embodiment of FIG. 3 — the implantable component 344. A difference between this embodiment and the embodiment of FIG. 4 is that the housing portion 632 housing the receiver-stimulator has a cubicle shape as opposed to a cylindrical shape. Also, continuing with the author’s keen eye for the obvious, the housing portion 318 that houses the electrical leads is located to one side of the housing portion 632. In this exemplary embodiment, the various components of the housing would be integral, but not monolithic. That said, in an exemplary embodiment, the housing portions 636, 632, and 618 can be potentially extruded and can be monolithic, while the lid 638 prevents the entire housing from being a monolithic body.

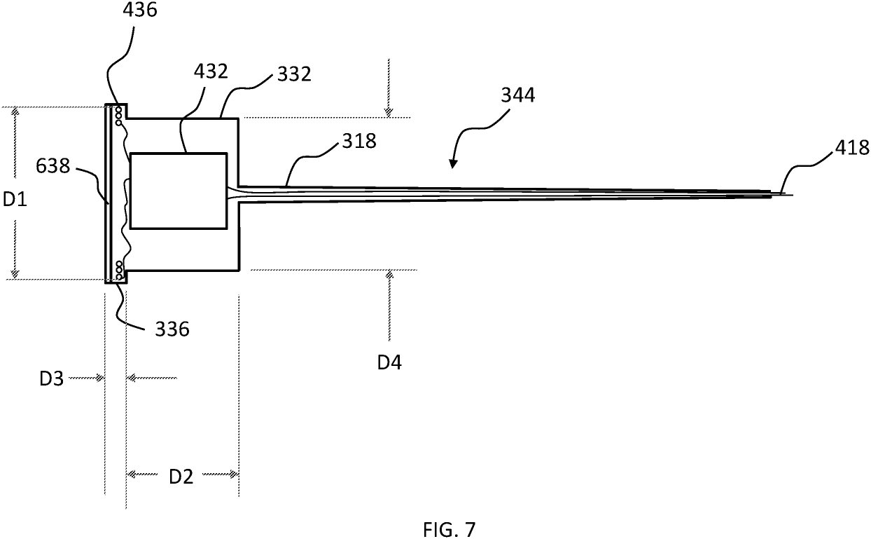

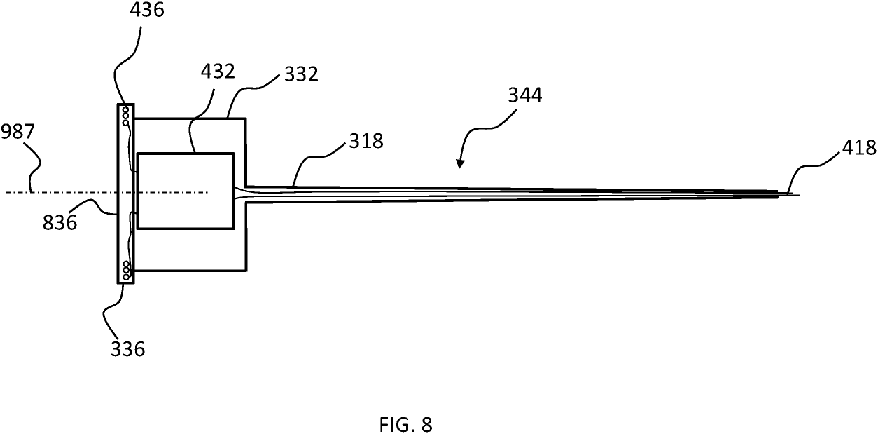

FIG. 7 presents a cross-sectional view of the embodiment of FIG. 3. It is also noted that this cross-sectional view can correspond in principle to the embodiment of FIG. 6 (the cross-sectional view would be taken against the far wall / the wall that supports the housing portion 618). Here, it can be seen that the coil 436 is within the same volume as the receiver-stimulator 432. This can be achieved owing to the utilization of the lid 638 that is transparent or at least sufficiently transparent to the inductance field generated by the external component so that the implanted coil 436 can receive the inductance field in a utilitarian manner. To be clear, in an exemplary embodiment, the interior of the housing is hermetically sealed. This can be achieved by utilizing a substance to seal the lid 638 to the titanium establishing the housing portion 436. In an exemplary embodiment, glass packaging technology could be used as an alternate to ceramic and/or titanium bodies. At the opposite end, the opening of the housing portion 318 can be plugged with a material that permits the electrical leads 4182 to extend therethrough, but prevents body fluids or the like from entering the housing, or at least from reaching the housing portion 332 (the leads 418 can be sufficiently individually insulated such that exposure to body fluids over the expected time periods of utilization do not result in a deleterious event in a statistically significant manner).

FIG. 8 depicts a two volume housing arrangement. Here, there is a housing 836 that establishes a separate self-contained volume relative to the volume established by housing portion 332, etc. In an exemplary embodiment, housing 836 can be a ceramic body that encases or otherwise encapsulates coil 436. A feedthrough or the like can be utilized to electrically connect the coil 436 to the receiver-stimulator 432. A feedthrough such as the feedthrough that is utilized to connect the coil of a standard implantable component of a cochlear implant to the receiver-stimulator within a housing of such standard implantable component can be utilized in this embodiment as well (or multiple feedthroughs, for that matter). Here, the electrical connection between the coil number 436 and the receiver-stimulator 432 is represented by two electrical leads.

In an exemplary embodiment, the ceramic housing 436 can be sealed to the titanium of the housing portion 332, or secured in any manner that can have utilitarian value. That said, in an exemplary embodiment, the housing 436 can be a composite housing that has a lid, such as the lids detailed above, that closes a titanium housing portion of the remainder of housing 836, and that titanium housing portion can be laser welded or the like to housing portion 332, where housing portion 332 is also titanium. In an exemplary embodiment, housing portion 332 can have a separate lid or the like or otherwise can have a separate “roof” that hermetically isolates the interior thereof despite the presence or absence of the housing 836. That roof could have a separate feedthrough or the like that will communicate with a feedthrough of the housing 836 to enable electrical communication between the coil 436 and the receiver-stimulator 432.

In an exemplary embodiment, one or more or all of the housing portions can be made of a ceramic material. In an exemplary embodiment, one or more or all of the housing portions can be made of glass or some other material that is transparent or otherwise effectively transparent to inductance field communication radiation. As will be described in greater detail below, the housing portion 336 can be a separate housing from the housing 332, and the utilization of metallic vias for a feedthrough can be utilized to communicate between the coil in the housing 336 and the housing 332. The housing portion can be made, at least in part, of a material that is transparent or effectively transparent to magnetic fields of an inductance coil.

In at least some exemplary embodiments, housing portions herein can be sealed so as to establish a hermetic enclosure utilizing laser welding or any other applicable technology.

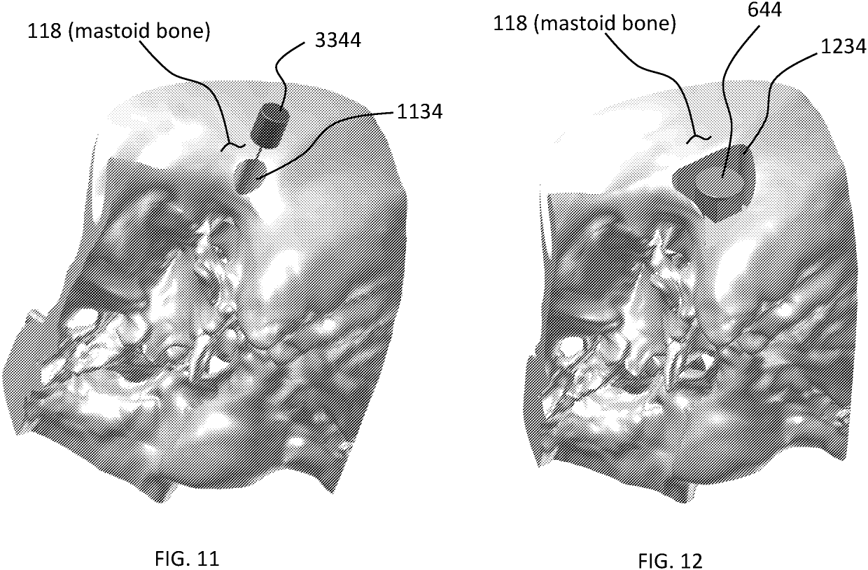

Briefly, to jump ahead with respect to implantation arrangements, the embodiments of FIGS. 3 to 8 are configured to be implanted into a skull of a recipient such that the longitudinal axis 987 of the coil 436 (See FIG. 8) / the axis about which the coil extends is closer to (including the same as) normal to the tangent plane of an extrapolated surface of the skull and/or the skin surface above the coil than parallel to such plane at the location where the skull bone is drilled or excavated to provide room for the implant. Additional details of this will be described below, but it is noted that in alternative embodiments, the axis 987 runs closer to parallel to that tangent plane as opposed to normal owing to the fact that the coil 436 is located proximate the external ear canal (the axis extends through the canal wall). Briefly, FIGS. 11 and 12 provide an exemplary implantation scenario, where FIG. 11 shows a circular drilled hole 1134 into the mastoid bone down to a transcranial tunnel, and FIG. 12 shows a limited suprameatal well 1234 extending to a transcranial tunnel. FIG. 11 also depicts another exemplary embodiment of an implantable component 3344 where the coil is located in the same housing portion as that which houses the receiver-stimulator, and the housing portion has a diameter that is governed by the coil diameter (described below). FIG. 13 depicts implantation of the alternate embodiment where the implantable component 944 is implanted such that the coil is proximate the ear canal.

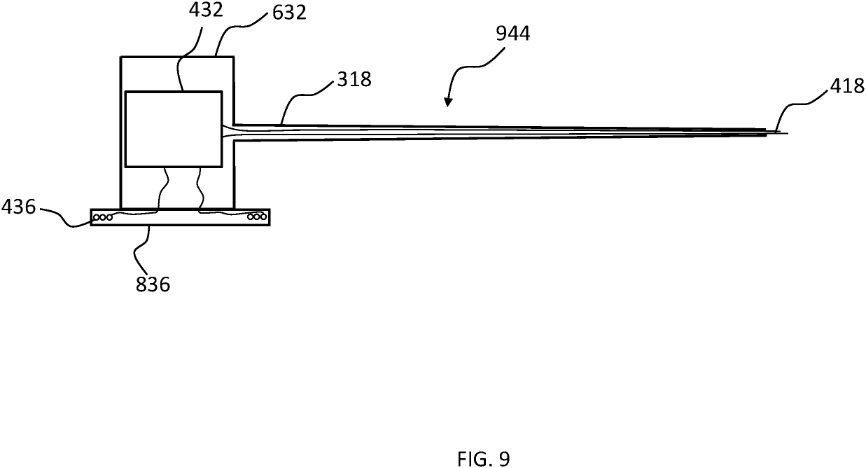

Again, additional details will be provided below, but the point is that in some embodiments, the placements of the coil with respect to the longitudinal axis of the overall implant can vary for different embodiments. In this regard, FIG. 9 presents an exemplary implantable component of a cochlear implant 944 that has the coil 436 located on a side of the implant as seen. Any placement of the coil and the associated housing/housing portions that can enable the teachings detailed herein can be utilized in at least some exemplary embodiments.

FIG. 10 presents another exemplary embodiment of the implantable portion of a cochlear implant 1044, which corresponds to the embodiment of FIG. 7 above, except that the housing portion 332 is replaced with the elongate lead assembly 22202. In this regard, as briefly noted above, some embodiments have a housing that stops at the right side of housing portion 332. This is an exemplary embodiment thereof. In this exemplary embodiment, the elongate lead assembly 22202 can correspond to the elongate lead assembly of a conventional implantable portion of a cochlear implant, albeit shortened in length in view of the implantation regimes that will be detailed below. The embodiment of FIG. 10 has a feedthrough and an elongate lead assembly mounting component 1057 that provides a physical connection for the elongate lead assembly 22202 and a feedthrough for the leads 418 that extend through the elongate lead assembly 22202. In an exemplary embodiment, the elongate lead assembly 22202 is physically connected or otherwise physically supported to the housing portion 332 in a manner that is the same as or otherwise similar to or otherwise analogous to that which is the case for a conventional implantable portion of a cochlear implant vis-à-vis the attachment of the elongate lead assembly to the housing of the receiver-stimulator, or in some embodiments, a variation thereof that is configured for the geometry of the housing portion 332.

The arrangement of FIG. 10 can have utilitarian value with respect to providing a greater range of positioning regimes associated with the portions distal of the housing portion 332. In this regard, by utilizing the flexible elongate lead assembly 22202 in lieu of the housing portion 318, the “maximum radius” of positioning of the electrode array 146 relative to a point on the housing portion 332 is greater and also provides more options (for example, instead of the radius of electrode array 146 positioning being controlled by the length of lead assembly that extends from the distal end of the housing portion 318, the radius of the electrode array 146 is controlled by the total length of the lead assembly from housing 332 (or the mounting component).

Further, the embodiment of FIG. 10 can have utilitarian value with respect to providing a single apparatus that can be utilized for multiple implantation regimes. For example, by simply resiliently bending the elongate lead assembly from the arrangement shown in FIG. 10 approximately 90° counterclockwise (relative to the plane of the page of FIG. 10), the implantable portion can be placed into a configuration for placement corresponding to that of FIG. 9 (proximate an ear canal). But also note that in at least some exemplary embodiments, the housing portion 318 can be sufficiently flexible and/or malleable to enable the housing portion to be bent and/or flexed by 40, 45, 50, 55, 60, 65, 70, 75, 80, 85, 90, 95, 100, or 110 degrees or any value or range of values therebetween in 1° increments (66, 88, 67 to 93 degrees, etc.). This can enable a single implantable portion to be utilized for both of the implantation regimes detailed above (longitudinal axis of the coil being normal to the plane of the skull vs. extending through the ear canal, as will be described in greater detail below).

As can be seen, in the embodiment of FIGS. 3 to 10, there is no separate cable or leads extending outside a housing that is exposed to the ambient environment to the coil. That is, because the coil is effectively hard mounted (i.e., not flexible in any meaningful way, as contrasted to, for example, a silicone body that supports the coil to a housing of the receiver-stimulator in the traditional implantable portion of the cochlear implant), direct feedthrough as can be utilized to communicate between the housings, if a feedthrough is even needed at all, can be achieved without the need of extra cables or extra leads extending from the feedthrough between a housing and/or outside of the housing to the cable. Indeed, with respect to the embodiments herein, movement of the housing portion 332 in a vector in any of the three dimensions always results to an equal movement of the coil along that vector as well, and vice versa, unless the implantable component is going to be significantly permanently deformed if not destroyed.

It is also noted that while the embodiments above disclose the coil having a maximum outer diameter that is larger than the maximum outer diameter of the housing portion 332, when the diameters are measured on parallel planes, in other embodiments, this may not necessarily be the case (it is noted that in other embodiments, the opposite can be the case). In an exemplary embodiment, the housing portion 332 can be configured such that the inner diameter is configured to accept in its entirety the inductance coil (the inner diameter will be larger than a maximum outer diameter of the inductance coil when measured on parallel planes).

Briefly, embodiments provide a relatively compact implantable component relative to the prior art. Briefly, by way of example only and not by way of limitation, FIG. 7 shall be utilized to present some exemplary dimensions. In an exemplary embodiment, a maximum outer diameter D1 of the coil of the implantable component and/or a mean, median and/or mode outer diameter D1 can equal to or be less than 7, 7.5, 8, 8.5, 9, 10, 11, 12, 13, 14, 15, 16, 17 or 18 mm or any value or range of values therebetween in 0.1 mm increments. The maximum diameter of the housing portion lying on a plane parallel to the plane upon which the coil extends that houses the coil can be those values plus an additional 0.4, 0.6, 0.8, 1.0, 1.2, 1.4, 1.6, 1.8, 2, 2.2, 2.4, 2.6, 2.8 or 3 mm or any value or range of values therebetween in 0.1 mm increments. In an exemplary embodiment, a maximum outer diameter D4 of the housing portion 332 (the cylindrical embodiment) measured on a plane that is parallel to the plane upon which the coil extends of the implantable component and/or a mean, median and/or mode outer diameter D4 can equal to or be less than 5, 5.5, 6, 6.5, 7, 7.5, 8, 8.5, 9, 10, 11, 12, 13, 14 or 15 mm or any value or range of values therebetween in 0.1 mm increments. In an exemplary embodiment, a maximum length D2 of the housing portion 332 (or with respect to the rectangular or prism embodiment) of the implantable component and/or a mean, median and/or mode of D2 can equal to or less than 5, 5.5, 6, 6.5, 7, 7.5, 8, 8.5, 9, 10, 11, 12, 13, 14, 15, 16 or 17 mm or any value or range of values therebetween in 0.1 mm increments, and D3, the height of the portion of the housing housing the coil 436, can be less than or equal to 0.3, 0.4, 0.5, 0.6, 0.7, 0.8, 0.9, 1, 1.2, 1.4, 1.6, 1.8, 2, 2.25, 2.5, 2.75 or 3 mm or any value or range of values therebetween in 0.05 mm increments. It is noted that some embodiments use a circular coil, and others can use different shaped coils, such as, for example, a 2 dimensional kidney shape, or an oval or racetrack shape, etc. All of these have a maximum diameter, it is just that the maximum diameter is not equal to all or most diameters. In an exemplary embodiment, the coil can be a shape that allows the coil to at least generally follow the shape of the pinna where it attaches to the skull. It is noted that in some embodiment, housing portion 336 could be shaped accordingly, and, in fact, housing portion 332 could be shaped accordingly.

With respect to the embodiment that utilizes a cubicle housing for the receiver-stimulator, the value D4 can be a length and/or a width (D2 would be the height), and the values of the two can be different from one another. They also be the same.

It is briefly noted that other embodiments can utilize a prism shaped stimulator-receiver housing portion that has 5, 6, 7 or 8 or more sides (e.g., octagon shaped cross-section, pentagon shape cross-section - the cross-section lying on a plane normal to the longitudinal axis of the housing portion. In an exemplary embodiment, the housing portion or otherwise housing proper of the coil 436 can be located on one of those flat sides of the prism a rectangle (or triangle for that matter). Indeed, the embodiment of FIG. 9 depicts a rectangular shaped housing 632 with the housing 836 is supported by one of the four sides of the housing 632.

It is briefly noted that the internal dimensions of the receiver-stimulator housing portion can be less than or equal to 150, 160, 170, 180, 190, 200, 210, 220, 230, 240, 250, 260, 270, 280, 290, 300, 310, 320, 330, 340, 350, 360, 370, 380, 390, 400, 410, 420, 430, 440 or 450 mm ³ or any value or range of values therebetween in 1 mm³ increments. To be clear, in an exemplary embodiment, the receiver-stimulator housing portion is the functional equivalent of the housing that is utilized for a receiver-stimulator of a traditional cochlear implant. It is also noted that in some embodiments, the housing where housing portion or a portion of the housing portion can be utilized as a return electrode. Alternatively and/or in addition to this, return electrode can be mounted on the wall of the housing portion.

FIG. 10 shows an exemplary dimension D11 of a distance from the end of the housing portion 332 to the distal most portion of the electrode array 146. While not specifically shown in the figure, in an exemplary embodiment, this dimension D11 is a length when the lead assembly 22202 and the electrode array 146 are extended to be essentially in a straight line, and otherwise extended to the point almost just below where further extension would result in plastic deformation (that is, the extension is such that once relieved, the lead assembly in the array will go back to its natural condition without any permanent deformation - we are simply trying to convey how one could measure this dimension D11 in a strict manner). In an exemplary embodiment, D11 is less than and/or equal to 10, 11, 12, 13, 14, 15, 16, 17, 18, 19, 20, 21, 22, 23, 24, 25, 26, 27, 28, 29, 30, 31, 32, 33, 34, 35, 36, 37, 3839, 40, 41, 42, 43, 44 or 45 mm or any value or range of values therebetween in 0.1 mm increments.

Thus, in view of the above, an exemplary embodiment includes an apparatus that includes implantable housing and implantable electronics hermetically sealed within the implantable housing. This apparatus further includes an implantable inductance coil. In this exemplary embodiment, an outer diameter of the inductance coil is less than 17.5 mm (or less than 15, etc., and he the values for D1 noted above). In an exemplary embodiment, this inductance coil is located in its entirety within 1, 2, 3, 4, 5, 6, 7, 8, 9 or 10 mm of portion(s) of the housing. In an exemplary embodiment, this apparatus is fully implantable within a human (as distinguished from a totally implantable hearing prostheses - here, we are explaining a component that is fully implantable and thus has no part that is outside of the recipient / human). Consistent with the teachings detailed above, in an exemplary embodiment, this apparatus is an implantable portion of a cochlear implant, and collectively, the coil and the at least the portion of the housing that houses the electronics have a geometry that fits within a 20 by 20 by 20 mm cube or, in some embodiments, a cube having length that is 10 to 25 mm or any value or range of values therebetween in .1 mm increments, a width that is 10 to 25 mm or any value or range of values therebetween in 0.1 mm increments, and a height that is 5 to 25 mm or any value or range values therebetween in 1 mm arguments.

In view the above, it can be seen that in some exemplary embodiments, there is a housing portion that houses electronics (receiver-stimulator, for example) of the implantable portion, and that housing portion has a length, a width and a height, wherein the height of the housing is at least 30, 35, 40, 45, 50, 55, 60, 65, 70, 75, 80, 85, 90, 95 or 100% or more or any value or range of values therebetween in 1% increments of the maximum diameter of the coil, where in the height is measured in a direction parallel to a longitudinal axis of the coil.

Thus, consistent with the teachings detailed herein, the apparatus is a miniaturized implantable portion of a cochlear implant. In at least some exemplary embodiments, at least a portion of the housing is implanted in a mastoid bone of a human recipient, and the coil is located above the electronics with respect to the direction above the surface of the mastoid bone and extends outboard of outer walls of the housing.

In an exemplary embodiment, the coil of the implantable component, or more accurately, the housing or housing portion of the implantable component that houses the coil, is the feature that drives the outboard dimensions of the implantable component. That is, with respect to looking down the longitudinal axis of the implantable component, or the longitudinal axis of the coil, the coil and/or the outer surface of the outer wall of the housing or housing portion that houses the coil, extends out beyond the outermost extension of the housing or housing portion that houses the electronics by at least or equal to 5, 10, 15, 20, 25, 30, 35, 40, 45, 50, 55, 60, 65, 70, 75 or 80% or any value or range of values therebetween in 1% increments of the total outside diameter of the housing portion that houses the electronics as measured on a plane that is parallel to the longitudinal axis of the coil, where the extension is also measured on that plane.

In an exemplary embodiment, this apparatus is configured for functional implantation in a recipient with the at least a portion of the housing implanted in the mastoid of a human skull, and the coil implanted in the recipient so that the coil is shadowed by a pinna of the recipient when viewed directly from the side of the human skull (the frame of reference of FIG. 14). Here, the coil is shadowed, and thus the coil is completely shadowed. In some other embodiments, only a portion of the coil is shadowed.

In some embodiments, the apparatus is in receptive wireless signal communication with a behind-the-ear device or an in-the-ear device worn behind an ear of a human or in an ear canal of a human, respectively, and the wireless signal communication emanates directly from a respective body of the BTE or ITE device. This as opposed to the arrangement detailed above where there is a headpiece connected to the body of the BTE device via a coil.

In at least some embodiments, the housing and coil are part of an integral assembly, wherein structure proximate the coil establishes a diameter, when measured on a first plane, that is at least as large as an outer diameter of the housing, when measured in a plane parallel to the first plane, the apparatus is magnetless.

Indeed, in an exemplary embodiment, there is no magnet whatsoever in the implantable portion and otherwise implanted in the recipient of the cochlear implant. In an exemplary embodiment, this can enable the recipient to be exposed to a 1, 1.5, 2, 2.5, 3, 3.5, 4, 4.5, 5, 5.5, or 6 T or greater MRI of the skull without requiring any presurgery or post surgery within one month of undergoing such MRI, such as presurgery or post surgery to remove a magnet. Accordingly, there is a method of having such an MRI in association with such lack of surgery within the aforementioned temporal periods. Accordingly, there is a method of having such an MRI in association with such lack of a magnet within the implant anywhere for a period of over one month before the MRI. In an exemplary embodiment, there is a method in the teachings herein can enable the application of the above noted MRIs without any restraints over the skin where the coil of the implant is located (e.g., no bandaging or utilization of splints). The recipient can go in and have them be exposed to an MRI of the skull without having to do anything except remove the external component as it relates to the prostheses. In an exemplary embodiment, there is no retention magnet in the implantable portion and/or no retention magnet in the human recipient, but there could be other magnets that are utilized for the operation of the implantable portion, such as a magnet of an electromagnetic actuator. In an exemplary embodiment, there is the method of having such an MRI in association with such lack of any retention magnet within the implant and/or within the body for a period of at least one month prior to the MRI. In an exemplary embodiment, there is the method of having such an MRI in association with such lack of any retention magnet within the implant or within the body ever before the MRI.

Some features of placement of the implantable components of the cochlear implants of the embodiments of FIGS. 3 to 10 will now be described.

In an exemplary embodiment, in a broad sense, the receiver-stimulator and the coil of the cochlear implant, or at least a portion thereof, is placed in / located in the suprameatal well that is established by a surgeon for cochlear implantation. This is opposed to the traditional method where the receiver-stimulator and the coils are located above the surface of the skull / in a skull excavation away from the suprameatal well. That said, in an alternative embodiment, at least a portion of the receiver-stimulator (or, more accurately, the housing portion thereof) is located in a suprameatal bore that is drilled using a drill. In this regard, in an exemplary embodiment, the difference between a well and a bore is that the latter has for the most part a circular cross-section because it is established utilizing a rotary drill which forms a cylindrical bore when such is utilized in the normal manner (as opposed to, for example, a ball shaped bone excavator or the like, where the tip of the rotary element is a spherical body that is utilized to “hog out” bone, which leaves a non-cylindrical bore). In an exemplary embodiment, a surgeon creates a suprameatal well having dimensions about the same as those of a standard Veria cochlear implant surgery. In an exemplary embodiment, the well that has, for example, a length of 15 mm, a width of 15 mm, and a depth of 20 mm is created in the skull of a recipient. Briefly, in an exemplary embodiment, the length of the well or bore can have a length of 5 to 15 mm, a width of 5 to 15 mm, and a depth of 5 to 20 mm, with any values or ranges of values within those ranges with 0.1 mm increments. In an exemplary embodiment, a total volume of the suprameatal well or bore that is established is less than or equal to 90, 85, 80, 75, 70, 65, 60, 55, 50, 45, 40, 35, 30, 25, 20, 19, 18, 17, 16, 15, 14, 13, 12, 11 or 10% or any value or range of values therebetween in 1% increments (66, 54, 72 to 33 %, etc.) of the volume of a standard suprameatal well. In an exemplary embodiment, the depth of the well will be governed by a the traditional depth required for otherwise utilitarian to reach the transcranial tunnel which extends from the middle ear cavity to the well, through which the elongate lead assembly or other corresponding portion of the implantable portion is extended from the well to the cochlea (adjacent the middle ear cavity).

In an exemplary embodiment, the suprameatal bore can have a depth corresponding to those above, and a diameter corresponding to the length or the width noted above, and can have a volume corresponding to those above.

Any bore or well having a size that can enable the teachings detailed herein can be utilized in at least some exemplary embodiments, specifically, the receipt of the implantable components of the cochlear implant noted above.

With respect to FIG. 14, there is a Cartesian coordinate system presented that is centered about the ear canal 106 (outer canal) of the recipient, and also a polar coordinate system centered about the ear canal 106. As can be seen, the Cartesian coordinate system is established by a vertical line 99 and a horizontal line 98 centered at the center of the ear canal 106 (the origin is the center of the ear canal 106, and has the coordinates 0, 0). The polar coordinate system is established utilizing the vertical line 99, the angle A1 and the distance D1 (the origin is the center of the ear canal). With respect to the schematic of FIG. 14, the plane of the schematic is taken at the location that passes through the outermost portion of the ear canal that has skin on all sides lying on a plane that is parallel to the orientation shown in FIG. 14 (for example, if the plane was located say a few millimeters closer to the viewer, the shape of the ear canal would look like a C-shape instead of a closed oval). All shapes are superimposed outlines that would be projected on that plane when viewed with the orientation of FIG. 14, but it is noted that the shapes may not be exactly as they appear in FIG. 14. More on this in a moment.

Shown in FIG. 14 is a suprameatal bore 1134 and/or implantable component 344. We utilize the same shape in FIG. 14 to represent both of these features owing to the fact that FIG. 14 is presenting spatial relationships, and in the embodiments that utilize component 344 and a bore, the former can be coaxial with the latter, or more accurately, the housing portion that houses the coil and/or the housing portion that houses the receiver-stimulator can be coaxial with the bore. As noted in the prior paragraph, the shapes of FIG. 14 may not be exactly as they appear. In this regard, for example, the longitudinal axis of bore 1134 may not necessarily be exactly normal to the plane of FIG. 14. It could extend obliquely to the plane of FIG. 14. Thus, in an exemplary embodiment, the shape representing the bore 1134, more accurately, the shape representing the top portion/outer portion of the bore, or the top of the implantable component 344, would be oval as opposed to circular. FIG. 14 presents dimensions with respect to center points of features that are projected onto the plane as defined above.

In an exemplary embodiment, X1 can be equal to or less than 4 to 30 mm or any value or range of values therebetween in 0.1 mm increments (e.g., 6, 8.83, 7.1 to 22 mm X1 can also be greater than or equal to -13, -6.5, or 0 mm, or any value or range of values therebetween in 0.1 mm increments. (Note that measurements to the right of line 99 are negative values.) In an exemplary embodiment, Y1 can be equal to or less than 0 to 30 mm, or any value or range of values therebetween in 0.1 mm increments (e.g., 2.2, 4.4, 3.4 to 29.3, etc.). Y1 can also be greater than or equal to -13, -7.5, or 0 mm, or any value or range of values therebetween in 0.05 inch increments (Note that measurements below line 98 are negative values). In an exemplary embodiment, D1 can be equal to or less than 5 to 20 mm, or any value or range of values therebetween in 0.1 mm increments. In an exemplary embodiment, A1 can be equal to or less than 10, 15, 20, 25, 30, 35, 40, 45, 50, 55, 60, 65, 70, 75, 80, 85, 90, 95, 100, 105, 110, 115, 120, 125, or 130 degrees, or any value or range of values in 1 degree increments. A1 can also be greater than -30, -25, -20, -15, -10, -5, 0, 5, or 10 degrees, or any value or range of values in 1 degree increments.

FIG. 15 depicts a side view of a human skull that has been annotated. As can be seen, the parietal bone 899 and the occipital bone 894 abut the mastoid bone 898. The zygomatic bone 895 and the mandible bone 893 are depicted to the right of the external acoustic canal 896 (ear canal). It is noted that the styloid process 897 is not considered to be part of the mastoid bone 898 for the purposes of this application. Superimposed upon the view of the skull is a borderline 801. Traveling from right to left, the borderline 801 extends along a horizontal line that has the same latitude (direction up and down with respect to the frame of reference of FIG. 15) as the junction of the temporal bone and the zygomatic bone. The borderline then follows the lower border of the mastoid bone to the location where the mastoid bone and the occipital bone and the parietal bone meet, and then extends along a horizontal line that has the same latitude as that meeting junction.

In an exemplary embodiment, with respect to a view looking in the frame of reference of FIG. 15, all of one or more of the bore, the well, the housing portion that houses the receiver-stimulator, the housing portion that houses the coil or the entire implantable component is located within the boundaries of area established by the curve 1577 and the two bounded vertical and horizontal lines and/or within the boundaries of curve 1515 and the two bounded vertical and horizontal lines, and/or within the boundaries of curve 1588 bounded by the two vertical lines and one horizontal line where FIG. 15 is drawn to scale and corresponds, in some embodiments, to a 50 percentile 30, 40, or 50 year old male Caucasian born in the United States of America, and in other embodiments, such a female, or in an exemplary embodiment, at least a portion of the housing portion that houses the coil and/or the housing portion that houses the receiver-stimulator is within the aforementioned areas.

In an exemplary embodiment, with respect to the aforementioned types of humans, at least a portion of the coil of the implanted inductance coil when viewed directly from a side of the head of the recipient is within one or more of the aforementioned boundaries (when viewed as noted above). In an exemplary embodiment, by area, at least 10, 15, 20, 25, 30, 35, 40, 45, 50, 55, 60, 65, 70, 75, 80, 85, 90, 95 or 100% of the area established by the outermost coil of the implanted coil is within the outer boundaries just detailed. Is also noted that the aforementioned features can also be applicable for the coil of the external component (more on this below) when viewed from the noted views.

It is noted that the curves of 1577, 1588, and 1515 are equally distant from the centerline of the canal 896.

FIG. 16 presents a side view looking downward with respect to the perspectives of FIGS. 14 and 15 (e.g., looking at the “top” of the skull). As can be seen, the plane of extension of the coil 436 is substantially parallel to the surface of the overlying skin 9118. Also as can be seen, the longitudinal axis 987 of the implantable component 344 when located in bore 1567 and otherwise fully seated therein is slightly offset from the axis 977 that is normal to the tangent plane 7118 established by the extrapolated surface of the mastoid bone 8118. In an exemplary embodiment, the difference between the two can be an angle of A2, where A2 can be equal to or less than 30, 25, 20, 15, 10, 9, 8, 7, 6, 5, 4, 3, 2, 1, or 0 degrees, or any value or range of values therebetween in 1° increments. In this regard, there is utilitarian value with respect to having the coil roughly parallel to the outer surface of the skin 9118, so that when the coil of the external component is located on the surface of the skin, the coils are relatively parallel to one another.

FIG. 17 presents a view looking down the ear canal of a recipient that has received an implantable component of a cochlear implant according to some of the embodiments detailed herein. In an exemplary embodiment, the closest portion of the housing portion that houses the coil 436 is within or equal to a distance X10 from an inner most surface of the skin of the ear canal (outer ear). In an exemplary embodiment, X10 is 2, 3, 4, 5, 6, 7, 8, 9, 10, 11, 12, 13, 14, or 15 mm, or any value or range of values therebetween in 0.1 mm increments. Consistent with the teachings detailed herein, the plane of the coil / the plane upon which the coil extends, such as the plane normal to the longitudinal axis 987, is substantially parallel with the longitudinal axis 1799 of the ear canal at that location (which extends into and out of the page of FIG. 17 / normal to the page). In an exemplary embodiment, an angle of offset relative to the angle that is perfectly normal is less than or equal to 1, 2, 3, 4, 5, 6, 7, 8, 9, 10, 11, 12, 13, 14, 15, 16, 17, 18, 19, 20, 21, 22, 23, 24, or 25 degrees or any value or range of values therebetween in 0.1° increments. Indeed, in an exemplary embodiment, the coil of the in-the-ear device (described below) that generates the inductance signal can also be canted relative to that longitudinal axis when the device is fully seated within the ear canal. That is, there can be utilitarian value in some embodiments to have coil planes that are not parallel to the longitudinal axis of the ear canal at that local location. Still, in some embodiments, the longitudinal axis of the coil 436 is normal to the longitudinal axis of the ear canal at that location. Tangent plane 1717 is a plane that is tangent to the surface of the ear canal at the location where the longitudinal axis 987 “exits” the skin as it travels into the ear canal from the housing of the coil 436. In an exemplary embodiment, the longitudinal axis 987 is normal to that plane, and in other embodiments can have an angle according to any one of the aforementioned angles just detailed.

FIG. 27 depicts a cross-sectional view of the arrangement of FIG. 17 on a plane that is parallel to and lying on the longitudinal axis 1799 of the ear canal 110. Here, the implantable component 944 is located in a suprameatal well 2772. The top portion of FIG. 27 represents the opening of the ear canal into the pinna. FIG. 27 has a dimension X 20. This dimension extends from the longitudinal axis 987 to a plane parallel to that longitudinal axis that is as far away from that longitudinal axis that still has a completely closed cross-section of the walls of the suprameatal well 2772. That is, if that plane was raised, say for example, a half inch or so above the plane shown, only the far portion of the well 2772 would be on that plane because of the sloping nature of the bone at that location. Conversely, a plane lying about a half inch below that would still have the completely closed cross-section. In an exemplary embodiment, X20 is 10, 11, 12, 13, 14, 15, 16, 17, 18, 19, 20, 21, 22, 23, 24 or 25 mm or any value or range of values therebetween in 0.1 mm increments.

FIG. 18 depicts an exemplary external component 342 in the form of a BTE device according to an exemplary arrangement. As seen, BTE device 342 includes element 330, which functionally and structurally can, in some arrangements, correspond to element 230 above, with exceptions according to the teachings herein, and thus corresponds to the spine of the BTE device. However, hereinafter, element 330 will be referred to by its more generic name as the signal processor sub-assembly, or sometimes the electronics component of the BTE device, or sometimes, for short, the signal processor, or sound processor subassembly, or sound processor for short (but that is distinguished from the processor therein, which processes sound / signals, and are also referred to as a sound processor or signal processor — this is the pure electronics portion of the overall signal processor subassembly, the latter having a housing and supporting other components), in some instances. As can be seen, attached thereto is element 270 which is thus a power component of the BTE device, which in some instances herein will be referred to as the battery sub-assembly, or the battery for short. The battery sub-assembly 270 is removably attached to the sound processor sub-assembly 330 via, for example, a bayonet connector.

In an exemplary arrangement, BTE device 342 is configured to function as an external component of a cochlear implant or an external component of a middle ear implant, or an external component of a DACS. It is not an implantable component and does not include implantable components, but it is configured to electromagnetically communicate with an implantable component. Embodiments include one or more or all of the teachings herein embodied in the device of FIG. 2C. For example, this device can have the in-the-ear component 250. Briefly, it is also noted that the in the ear component 250 can be in wireless communication with the sound processor sub-assembly 330. That is, instead of the cable 252, a wireless link can be present. Some additional details of this feature will be described below with respect to another embodiment, and it is briefly noted that any of the teachings detailed below with regard to this other embodiment that utilizes the wireless link can be utilized with respect to the embodiment of FIG. 18 where the in the ear component is utilized to support a microphone.

It is noted that the teachings detailed herein and/or variations thereof can be utilized with a non-totally implantable prosthesis. That is, in some arrangements, the cochlear implant is a traditional hearing prosthesis.

In this exemplary embodiment, instead of the headpiece detailed above with respect to the embodiment of FIG. 2C, as can be seen, the sound processor sub- assembly 330 includes an inductive coil 1836. The inductive coil 1836 is located on the side of the “spine,” the part that faces towards the skin of the recipient lying over the mastoid bone / skull bone and facing away from the pinna when worn behind the ear. The coil 1836 can be located in the housing wall or just behind the housing wall of the spine. In an exemplary embodiment, the coil 1836 is substantially identical including identical in size and configuration to the coil of the implantable component, and interfaces therewith. In this embodiment, the coil 1836 takes the place of the coil in the headpiece of the embodiment of FIG. 2C. In this embodiment, the circuitry and components that are utilized to drive in otherwise power the coil 1836 are identical or otherwise analogous to the components that are utilized to energize or otherwise power the coil of the headpiece. In this regard, the coil 1836 is a substitute for the headpiece and otherwise eliminates that feature.

Accordingly, in an exemplary embodiment, there is a behind the ear sound processor without a separate coil apparatus/without a headpiece that is fully functional to transcutaneous communicate via an inductance field with an implantable portion of a hearing implant. In an exemplary embodiment, all inductance field communication components are located within, no more than 0.25, 0.5, 0.75 or 1 inch from the housing wall of the spine of the BTE device and there is no inductance field communication component further than 0.25, 0.5, 0.75 or 1 inch from the housing wall of the spine of the BTE device.

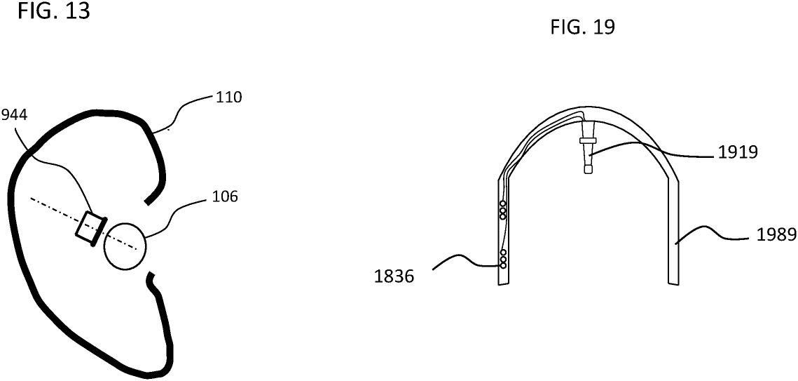

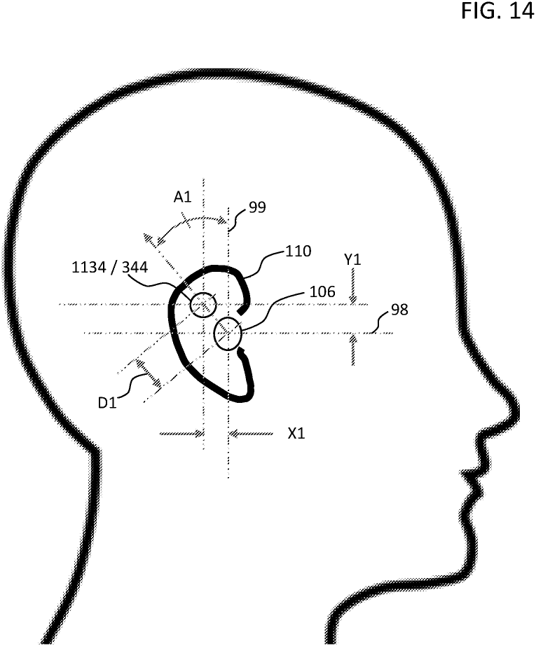

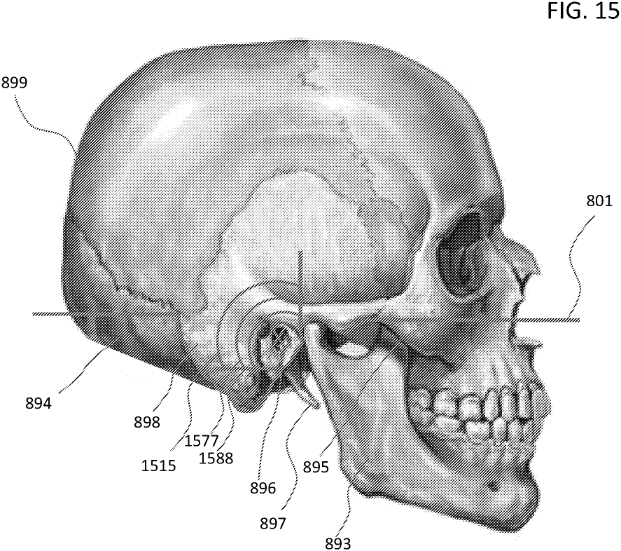

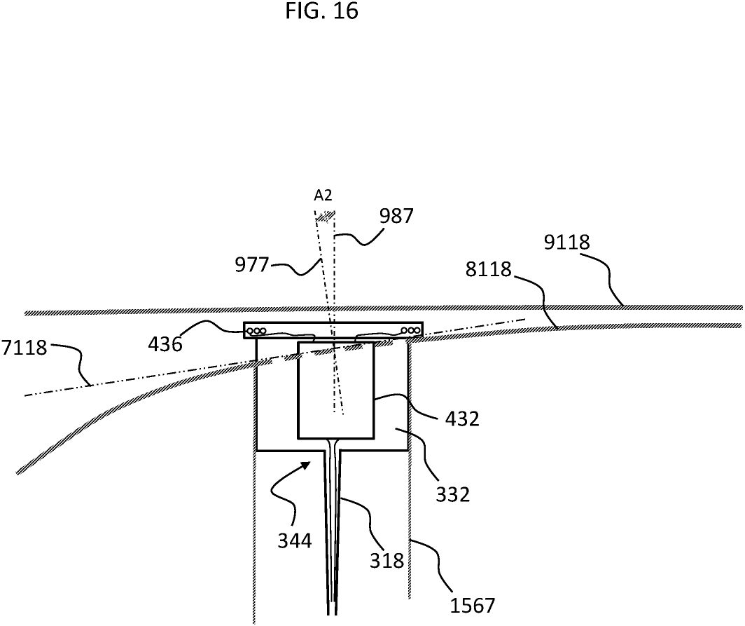

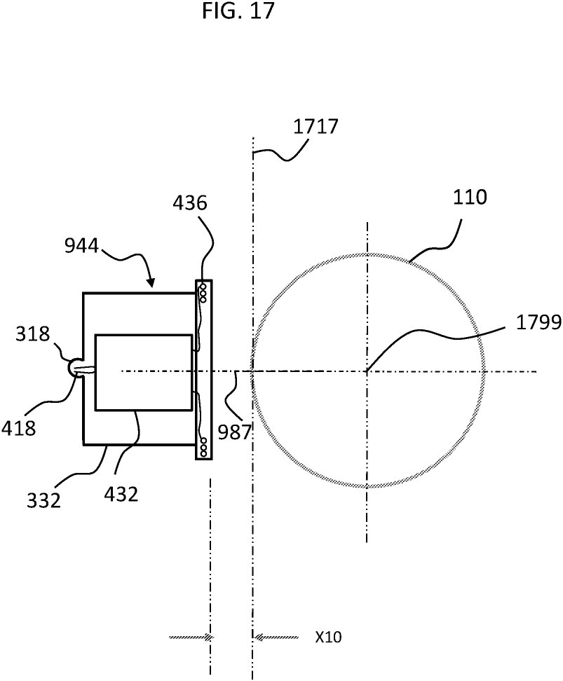

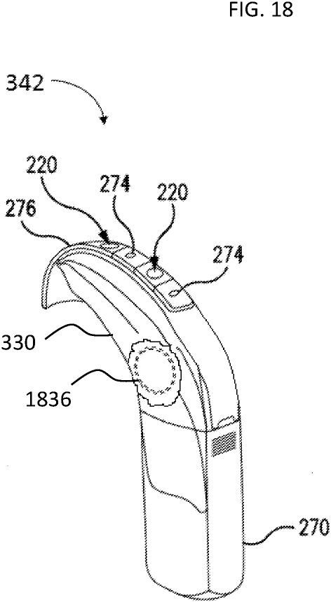

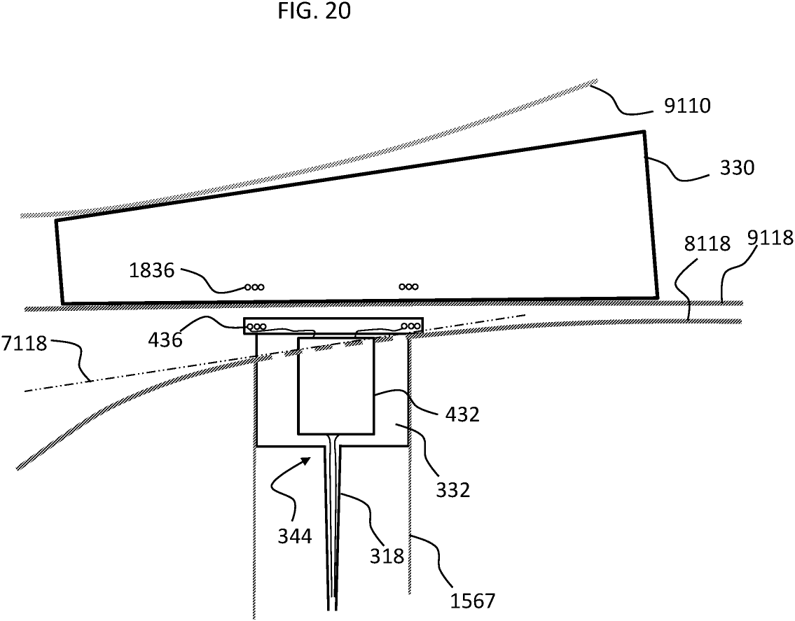

Briefly, some exemplary embodiments include retrofitting the arrangement of FIG. 2C to a configuration corresponding to that of FIG. 18. By way of example, FIG. 19 presents an exemplary saddle device that has a male jack portion 1919 that fits into the receptacle for the jack of the headpiece. An inductance coil 1836 is embedded in the saddle body 1989. In an exemplary embodiment, the headpiece is removed by removing the male jack that is attached to the cable connecting the jacket to the headpiece, and the saddle device is replaced over the spine, with one side of the saddle extending to the location where the coil 1836 is depicted in FIG. 18, wherein the saddle supports a coil like coil 1836, but the coil is located on the outside of the housing wall and supported by the saddle. In this way, a pre-existing behind the ear device that utilizes a traditional headpiece can be converted to a device that can be utilized the teachings detailed herein. The body of the saddle 1989 can be flexible and can resilient lay snapped coupled to the spine, and the jacket 1919 can provide further coupling to secure the saddle assembly in place.

Accordingly, in an exemplary embodiment, there is a method that includes retrofitting a traditional behind the ear device for a cochlear implant or for a middle ear implant or for a DACS implant that includes a headpiece connected to the spine / signal processor via a jack that is readily removable, by removing that jack and thus the headpiece and the associated coil from the body of the BTE device, and replacing that assembly with the saddle assembly FIG. 19, where the output to the saddle assembly can be the exact same as that which would be outputted to the headpiece.

FIG. 20 presents a view looking downward from the top of a recipient’s head (at the top of the head) showing the spine 330 located between the skin surface 9118 of the skin over the mastoid bone and the surface of the pinna 9110 facing the skin over the mastoid bone. As can be seen, coil 1836 is aligned with coil 436. In this exemplary embodiment, the coil drivers of the sound processor sub-assembly 330 located in the spine 330 drive the coil 1836 to generate an inductance field that is received by coil 436. That field that is received by coil 436 induces a current therein, which current is provided to the receiver-stimulator 436 of the implantable portion 344, which then drives the electrodes of the electrode array thereof to evoke a hearing percept in a manner corresponding to that of a cochlear implant.

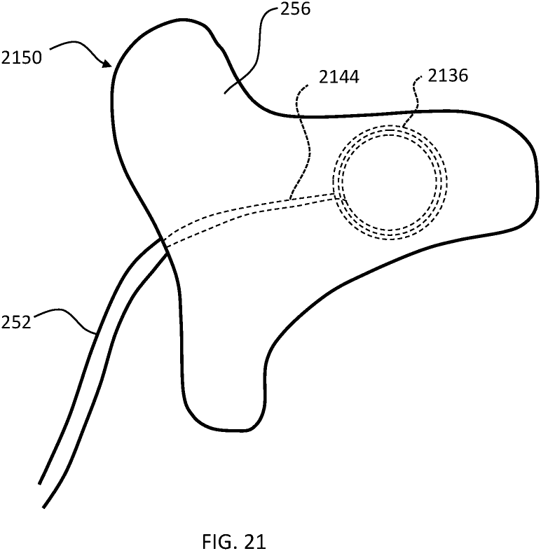

As briefly mentioned above, embodiments of the behind the ear devices that do without the headpiece for the inductance communication can utilize an in the ear component. In this regard, FIG. 21 depicts an exemplary in the ear component 2150 that can be linked to the spine/sound processor sub- assembly 330 via cable 252 (in the manner that the in-the-ear device 250 is attached to the spine of the embodiment of FIG. 2C above). In this exemplary embodiment, the cable 252 contains leads 2144 that extend to the inductance coil 2136. In this exemplary embodiment, instead of the inductance coil being supported by the spine 330, the inductance coil is supported by the in the ear component 2150. While this embodiment does not show the microphone, it is noted that in some embodiments, the in-the-ear device 2150 can have a microphone in a manner corresponding to the microphone of the in-the-ear device 250 detailed above in FIG. 1F. It is noted that the behind the ear device can also include a microphone 220 on the spine 330 even if there is a microphone in the in-the-ear device.

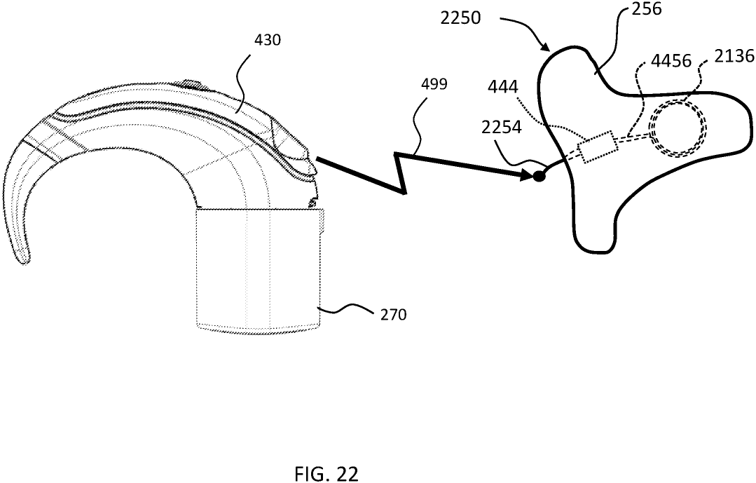

FIG. 22 presents an alternate exemplary embodiment of an external component of a hearing prostheses where in-the-ear device 2250 corresponds in functionality to the in-the-ear device 2150 detailed above, but the in-the-ear device 2250 is only in signal communication with the signal processor 430 (which can correspond to the signal processor 330 detailed above, at least with respect to the functionality thereof) by an RF link 499. Here, the in-the-ear device includes antenna 2254 which is in wired communication with receiver electronic apparatus 444, which converts the signals received by the antenna 2254 into usable signals that are usable by the coil 2136, wherein leads 4456 extend from the receiver 444 to the coil 2136. It is noted that in embodiments where element 2250 includes a microphone, element 444 can be a receiver transmitter, or there can be a separate transmitter to transmit the signal based on the output of the microphone to the signal processor subassembly 430 so that the signal processor subassembly can process the signal, and then output a signal to element 2250. Accordingly, in this exemplary embodiment, link 499 would be a two-way link.

In the exemplary embodiment of FIG. 22, the sound processor subassembly 430 can correspond to that detailed above vis-à-vis sound processor 330, with the exception that it would have the functionality just detailed accordingly, in this exemplary embodiment, the sound processor 430 can be configured to output an RF signal. In this exemplary embodiment, the outputted RF signal could have a power that it could not extend meaningfully into the skin of the recipient to power or control or otherwise operate an implanted component.

It is briefly noted that the arrangement of FIG. 22 can also be representative of an arrangement where the in-the-ear device is only used to support a microphone, and the inductance coil is located on the spine. The link can simply transmit the signal captured by the microphone to the sound processor sub- assembly 330 or 430.

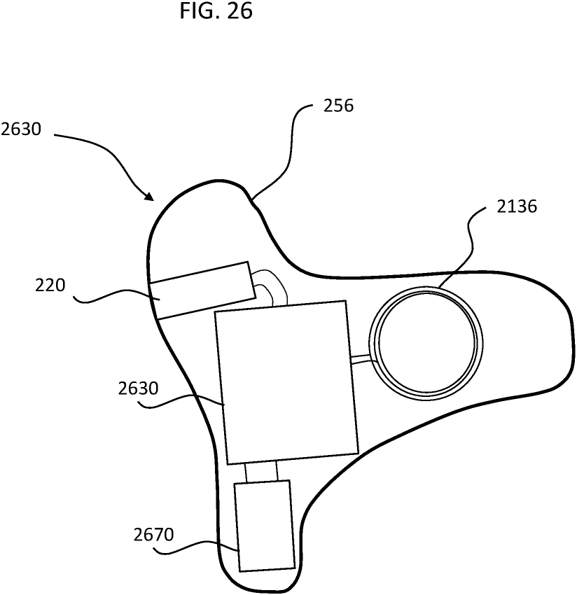

FIG. 25 presents a view looking down the longitudinal axis of the ear canal at the location where the coils of the implantable component and the external component are located. (It is noted that something that is in the ear canal is not considered to be implanted and is not considered to be underneath the skin of the recipient.) As can be seen, there is a suprameatal well 1234 in which is located the coil 436 (and other parts of the implantable component). Also as can be seen is the housing of the in-the-ear device 256 and the coil 2136 of that device (and the receiver 444 — it is noted that the receiver 444 is shown in phantom lines owing to the fact that this view is a cross-sectional view on a plane lying on the longitudinal axis 987 and normal to the longitudinal axis of the ear canal 110 at that location - the receiver 444 would be located above the plane shown in FIG. 25 — indeed, the sides of the receiver can be a size that is larger such that it would not necessarily fit into the location at the ear canal shown).

As seen, the distance from the surface of the inner skin of the ear canal lying on the longitudinal axis of the coil 2136 can be a distance X10. As seen, the plane 2536 upon which the coil 2136 extends is parallel to the plane 2599 upon which the coil 436 extends. In an exemplary embodiment, an angle between the two in one or two axes can be less than or equal to 1, 2, 3, 4, 5, 6, 7, 8, 9, 10, 11, 12, 13, 14, 15, 16, 17, 18, 19, 20, 21, 22, 23, 24, or 25 degrees, or any value or range of values therebetween in 1° increments. Also, it is noted that in an exemplary embodiment, the offset between the two longitudinal axes of the two coils can be less than or equal to 0.1, 0.2, 0.3, 0.4, 0.5, 0.6, 0.7, 0.8, 0.9, 1.0, 1.25, 1.5, 1.75, 2, 2.25, 2.5, 2.75, 3, 3.25, 3.5, 3.75, 4, 4.25, 4.5, 4,75, or 5 mm, or any value or range of values therebetween in 0.1 mm increments.

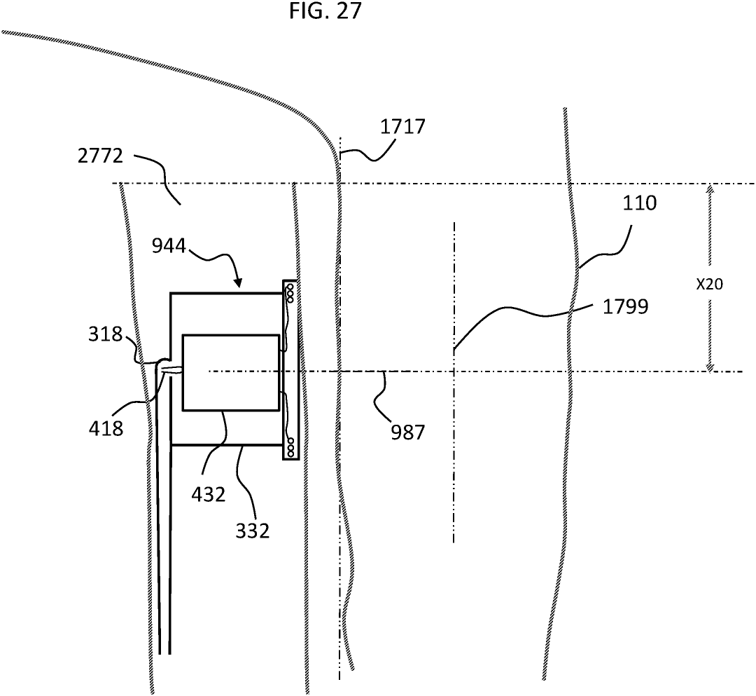

FIG. 26 presents another exemplary embodiment of an in-the-ear device 2630 according to the teachings herein. This device is a fully contained external component of a cochlear implant or a middle ear implant or a DACS. In this exemplary embodiment, the microphone 220 is supported by housing 256 which is in signal communication via leads to a sound processor 2630. In an exemplary embodiment, the sound processor 2670 can be a miniaturized version of the sound processor utilized with the embodiments detailed above, and can be a commercially available sound processor that is configured for utilization within an ITE device. As seen, there is a battery 2670 that provides power to the system. Consistent with the teachings above, there is an inductance coil 2134 that is in signal communication via leads with the sound processor 2630. In this exemplary embodiment, the ITE device 2630 inductively communicates with the implanted component in a manner concomitant with the teachings detailed herein with respect to the ITE device that is in signal communication with a BTE device.

In view of the above, it can be seen that in an exemplary embodiment there is a system, comprising an implantable component configured to be fully implantable in a human, such as the implantable components, 344 or 1044, etc., detailed above. Further, the system includes an external component, such as by way of example only and not by way of limitation, the external component 342 or the combination of elements 430 and 2250, configured to be, in its totality, retained on a head of the human via at least one of behind-the-ear structure or in-the-ear structure. In this regard, it is meant that the retention can be established via these techniques, as opposed to other techniques, such as, for example, a headband or a magnet (some prior art prostheses utilize an implanted magnet that interacts with a magnet of the external component to retain the external component against the skin of the recipient). This does not rule out the utilization of other techniques. It simply means that even if those other techniques were not present, the external component would be retained utilizing those techniques if those techniques were the only thing being used to retain the apparatus. By rough analogy, a hybrid car can be powered by both electricity from the battery and from gasoline. The hybrid car is configured to run solely on gasoline even though it can also run on electricity. Still, in some exemplary embodiments, the system is devoid of retention magnets / magnets that are used for retention, adhesives, headbands, etc. Accordingly, some embodiments explicitly exclude these retention mechanisms indeed, in an exemplary embodiment, the only retention mechanism that is present is a result of the behind the ear structure and the in-the-ear structure.

Further with this exemplary embodiment, consistent with the teachings detailed above, the external component is configured to be in inductance signal communication with the implantable component.

In an exemplary embodiment of the system under detail, the system is configured such that the inductance signal communication is maintainable through skin of the recipient human in the absence of magnetic attraction between the implantable component and the eternal component when the system is subject to a 0.5, 0.75, 1, 1.25, 1.5, 1.75, 2, 2.5, 3, 3.5, 4, 4.5, or 5 G acceleration, or any value or as a range of values therebetween in 0.05 G increments in one or more directions or in some embodiments, any direction tangent to a skin surface at the site of inductance signal communication. By any direction, it is meant that if the acceleration is directed in any angle over 360°, there is retention.

By way of example only and not by way of limitation, with respect to the embodiment where the coil is located at a location that is within the boundaries of the pinna for example / over the mastoid bone, the downward force of gravity would not dislodge or otherwise disrupt the inductance communication. Further by way of example only and not by way of limitation, with respect to the embodiment where the coil is located within the in-the-ear device, the associated friction forces between the skin of the ear canal and the device would prevent the device from being dislodged of the person were to lay on his or her side the year having the device facing downward, but sufficiently raised from another surface so that there is nothing other than skin of the ear canal and pinna contacting the device.

In this regard, in the system under detail, in an exemplary embodiment, the external component includes an in-the-ear device including a housing configured to be positioned in an ear canal of the recipient, and an inductance coil is located in and/or on the housing, which inductance coil establishes the inductance signal communication with the implantable component in conjunction with an implantable coil of the implantable component. In an exemplary embodiment, this inductance coil is located on a lateral side of the housing of the in-the-ear device, as opposed to the end side which faces the tympanic membrane when inserted into the outer ear, or the end side which faces the outside world when inserted into the outer ear. Still further, consistent with the teachings above, in an exemplary embodiment, the housing is configured to be located in an ear canal of the recipient and retained by friction forces therein, and the housing and coil are arranged so that the coil is located proximate and faces a sidewall of the ear canal when the housing is so located. This as opposed to, for example, an arrangement where the coil faces the tympanic membrane. In an exemplary embodiment, the plane upon which the coil extends in a direction that is parallel to a longitudinal axis of the ear canal, as contrasted to a coil that faces the tympanic membrane, which plane would be normal to a longitudinal axis of the ear canal.

Consistent with the teachings herein, the system can be a partially implantable cochlear implant, and the implantable component that is the implantable component of the partially implantable cochlear implant. That said, in some embodiments, the system can be a partially implantable middle ear implant, and the implantable component that is the implantable component of the partially implantable middle ear implant. That said, in some embodiments, the system can be a partially implantable DACS implant, and the implantable component that is the implantable component of the partially implantable DACS implant. With respect to these two latter embodiments, the actuator of the middle ear implant would be located in the middle-ear cavity, with leads extending therefrom through the transcranial tunnel to the bore or well, and then to the receiver-stimulator housing portion (or the equivalent to the housing that houses the electronics that drive the actuator — in this regard, there will be some form of device / circuitry, which is readily available in the art, in a given size or a size that is modifiable for the teachings detailed herein, that receives the inductance signal from the implanted coil, and automatically converts this signal to a drive signal or otherwise generates drive signal based on this signal received from the coil, that is outputted via leads the actuator — this can be a chip or a circuit configured to do so and is known in the art — any device system and/or method that can enable this can be utilized in some embodiments — in general, the receiver-stimulator or the analogous structure (perhaps a better name is a receiver-driver) of a middle ear implant that is commercially available presently or in a modified form can be utilized). With respect to the DACS implant, the leads extending from the receiver-stimulator (or receiver-driver) housing portion extend through the transcranial tunnel to the middle ear cavity and then to the cochlea wall and then potentially to inside the cochlea wall, where the actuators are located to stimulate the interior of the cochlea.

In an exemplary embodiment, the implantable component includes an inductance coil that establishes the inductance signal communication with the external component in conjunction with an external coil of the external component and the implantable component is configured to be implanted in the recipient such that an outer profile of a pinna of a 50 percentile male or female of 30, 40, or 50 years of age born in the United States of America encompasses the footprint of the coil of the inductance coil when viewed directly from a side of the head of the recipient. This is the embodiment of FIG. 14 as depicted if that figure was drawn to scale. In this regard, the generally C-shaped line drawling of the pinna 110 establishes the outer profile of a pinna, and as can be seen, the housing portion that houses the coil is entirely within the outer profile (elements 1134 / 344 can correspond to the area established by coil 436). It is noted that the alternate embodiment of the implantable component where the coil of the implantable component faces the ear canal (from the outside — as opposed to coil 436 when used in the embodiment of FIG. 3, where the coil does not face the ear canal and/or the plane upon which the implanted coil extends is at least about normal to the direction of extension of the ear canal) also meets this requirement and at least some exemplary embodiments. In an exemplary embodiment, with respect to the aforementioned types of humans, at least a portion of the coil of the inductance coil when viewed directly from a side of the head of the recipient is within the outer profile of a pinna. In an exemplary embodiment, by area, at least 10, 15, 20, 25, 30, 35, 40, 45, 50, 55, 60, 65, 70, 75, 80, 85, 90, 95 or 100% of the area established by the outermost coil of the coil is within the outer boundaries of the pinna with respect to the aforementioned humans. Is also noted that the aforementioned features can also be applicable for the coil of the external component. By way of example only and not by way of limitation, FIG. 14 can represent the coil / the area of the coil established by the outermost winding of the external component (elements 1134 / 344 can correspond to the area established by coil 1836).

In an exemplary embodiment, the external component of the above noted behind-the-ear device includes a housing configured to be positioned behind-the ear of the recipient. This housing can correspond to the housing of the spine 330 detailed above. In an exemplary embodiment, an inductance coil is located in and/or on the housing, which inductance coil establishes the inductance signal communication with the implantable component in conjunction with an implantable coil of the implantable component. In an exemplary embodiment, as can be seen from the FIGS. 18 and 20, the inductance coil is located on a lateral side of the spine, which lateral side faces the skin above the mastoid bone opposite the pinna in at least some exemplary embodiments. This as opposed to a coil that is located on the “roof” of the spine or the belly of the spine (the roof is where, for example, element 274 is located).

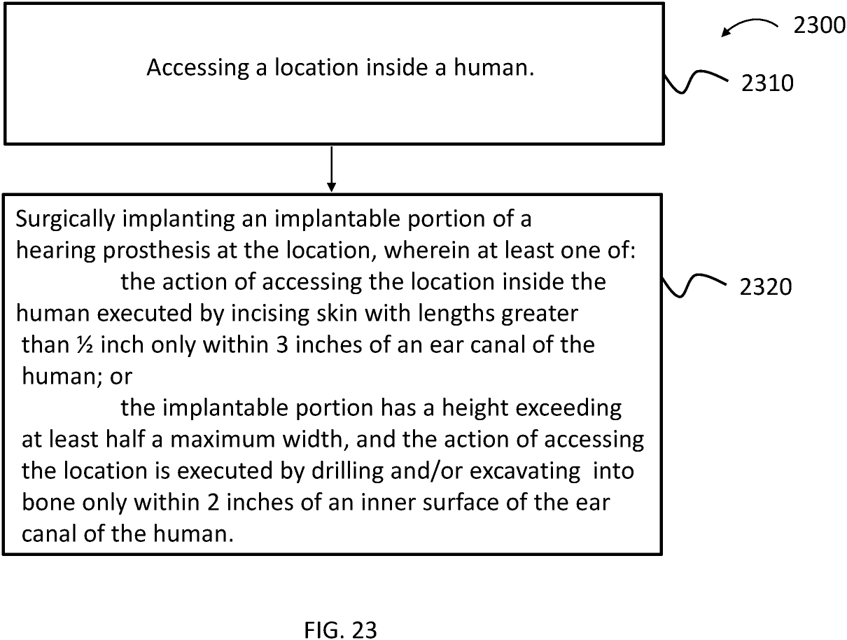

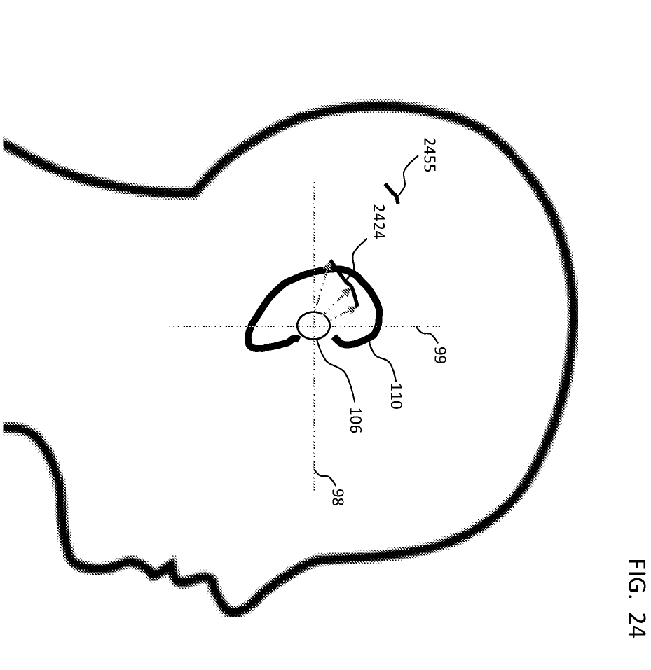

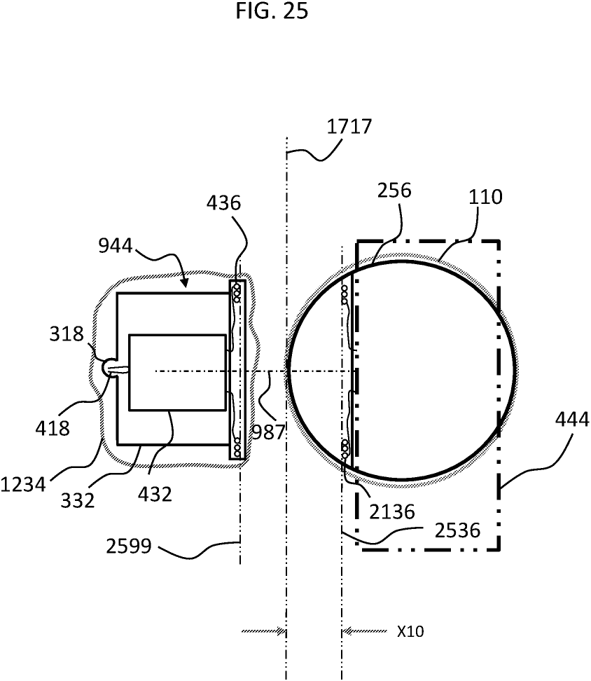

Embodiments include surgical methods. In this regard, FIG. 23 presents an exemplary flowchart for an exemplary method, method 2300, which includes method action 2310, which includes accessing a location inside a human. This can be accomplished by utilizing a surgical scalpel and cutting into the skin posterior to the ear canal / under the pinna (under in terms of the outline of the pinna with respect to the views of FIG. 14 encompassing at least a portion of this cut) and also utilizing a drill or a bone excavation device to drill the bore or excavate the suprameatal well to the location, which location can be a location having a depth from the surface pre-drilled/pre-excavated skull bone to the location of the transcranial tunnel, for example. Method 2300 further includes method action 2320, which includes surgically implanting an implantable portion of a hearing prosthesis at that location. In an exemplary, at least one of (1) the action of accessing the location inside the human is executed by incising skin with lengths greater than ½ inch only within 0.5, 0.75, 1, 1.25, 1.5, 1.75, 2, 2.25, 2.5, or 3 inches, or any value or range of values therebetween in 0.1 inch increments of an inner surface (meaning the skin surface) of an ear canal of the human, or (2) the implantable portion has a height exceeding at least half a maximum width, and the action of accessing the location is executed by drilling and/or excavating into bone only within 2 inches of an inner surface of the ear canal of the human.

With respect to the first scenario, referring to FIG. 24, it can be seen that there is an incision 2424 that is in large part behind the pinna 110 / within the boundaries of the pinna 110. This incision is greater than 1 inch in length (by measuring the path of the cut from one end to the other — not a linear distance (unless the incision is linear) but an actual distance — by analogy, the distance between New York and Miami can be a first value if using Interstate 95, and many times that first value if using Interstate 70 to Denver, and then traveling south to Texas, and then following the Gulf Coast and then cutting across to Interstate 95. It is the actual distance, not the geographic distance). Also, the above-noted distances from the inner surface of the ear canal correspond to any portion of that incision. This is exemplified by the three different arrows extending from the surface of the ear canal 106. The longest arrow is the lowest arrow, and thus the longest distance, and if that distance did not fall within, for example, 3 inches, that would mean that the incision did not fall within the above arrangement.

FIG. 24 also presents an incision 2455. This incision is less than ½ inch in length. Thus, even though it would exceed for example, 3 inches from the inner surface of the ear canal, because it is length is less than ½ inch, this still meets the above noted arrangement. If that exceeded ½ inch, then that would not meet the above noted arrangement.

Of course, in an exemplary embodiment, there are no incisions of any type that fall outside the above-noted distance from the ear canal, at least with respect to the action of accessing the location inside a human.

In an exemplary embodiment, there is only one incision made to access that location, and the length of that incision is less than 0.5, 0.75, 1, 1.25, 1.5, 1.75, 2, 2.25, 2.5 or 3 inches or any value or range of values therebetween in 0.1 inch increments. Indeed, in an exemplary embodiment, method 2300 is executed with the action of making an endaural incision, wherein the endaural incision extends no more than 0.5, 0.75, 1, 1.25, 1.5, 1.75, 2, 2.25, 2.5 or 3 inches, and wherein the endaural incision is the largest incision made during the surgical method. In an exemplary embodiment of the method, the method includes making an endaural incision, wherein the endaural incision is a distinct incision such that during the action of surgically implanting the implantable portion, the distinct incision is no longer than 0.5, 0.75, 1, 1.25, 1.5, 1.75, 2, 2.25, 2.5, or 3 inches or any value or range of values therebetween in 0.1 inch increments. By “distinct incision,” it is meant that any incision that extends to the endaural incision / is an extension of any initial endaural incision, is also part of that endaural incision. By way of example only and not by way of limitation, if the initial incision is an endaural incision, and then there is some other incision that is made it is not strictly classified as an endaural incision, but that incision extends to the initial endaural incision (thus “merging” the two), that becomes part of the distinct incision that includes the endaural incision.

With respect to the second scenario of method 2300, the implantable portion has a height exceeding at least half a maximum width, and the action of accessing the location is executed by drilling and/or excavating into bone only within 2 inches of an inner surface of the ear canal of the human, the explanations herein are also applicable to evaluating this feature with respect to the teachings above and below with respect to the various spatial features. In a variation of the method, the implantable portion has a height at least or equal to 0.4, 0.5, 0.6, 0.7, 0.8, 0.9 or 1 times or any value or range of values therebetween in 0.01 increments the maximum width, and the action of surgically implanting the implantable portion is executed by drilling and/or excavating into bone only within 0.25, 0.5, 0.75, 1, 1.25, 1.5, 1.75, 2, 2.5 or 2.5 inches or any value or range of values therebetween in 0.01 inch increments of an inner surface of the ear canal of the human, the explanations herein are also applicable to evaluating this feature with respect to the teachings above and below with respect to the various spatial features. In an exemplary embodiment, the method is executed such that the drilling and/or excavating into the bone occurs only within the various areas detailed above bounded by curves 1515, 1577 or 1588 with respect to FIG. 15. In an exemplary embodiment, the excavation and or drilling can have the limitations detailed herein with respect to the incisions and/or with respect to the locations of the coil and/or implantable component as detailed herein.

In some embodiments, the action of surgically implanting the implantable portion is executed with the recipient under only local anesthesia and sedation. In an exemplary embodiment, from the time at the beginning of commencement of making the first incision (i.e., just when the tip of the scalpel pierces / enters below the surface of the skin) to the point where the first incision can be closed (that does not mean that closing has commenced — it is the medical time where a surgeon can declare the incision ready for closure — whether that actually happens is a different issue), a time that has elapsed is no more than 20, 25, 30, 35, 40, 45, or 50 minutes or any value or range of values therebetween in 1 minute increments. That said, the above time frame can actually be defined at the end by the time point where closure actually commences.

In some embodiments the surgical implantation method is executed such that upon closure, all implanted components are within 0.5, 0.75, 1, 1.25, 1.5, 1.75, 2, 2.25, 2.5, or 3 inches, or any value or range of values therebetween in 0.1 inch increments of a skin surface of the ear canal.

In an exemplary embodiment, the implanted hearing prosthesis of the method 2300 is cochlear implant. In exemplary embodiment, the implanted hearing prosthesis of the method 2300 is a middle ear implant. In an exemplary embodiment, the implanted hearing prosthesis of the method 2300 is a DACS.

In an exemplary embodiment, the action of accessing a location inside the human is executed by creating a suprameatal well or a suprameatal bore, and the action of surgically implanting the implantable portion of the hearing prostheses includes placing the receiver-stimulator or the receiver-driver of the hearing prostheses at least partially within the well or bore. In an exemplary embodiment, by volume, at least or equal to 10, 15, 20, 25, 30, 35, 40, 45, 50, 55, 60, 65, 70, 75, 80, 85, 90, 95, or 100%, or any value or range of values therebetween in 1% increments of the receiver-stimulator or receiver-driver circuitry are located within the well or bore.

In an exemplary embodiment, with respect to a view looking in the frame of reference of FIG. 15, all of incisions meeting method 2300 or all of the incisions of the method are located within the boundaries of area established by the curve 1577 and the two bounded vertical and horizontal lines and/or within the boundaries of curve 1515 and the two bounded vertical and horizontal lines, and/or within the boundaries of curve 1588 bounded by the two vertical lines.

It is also noted that any disclosure herein of any process of manufacturing other providing a device corresponds to a device and/or system that results there from. It is also noted that any disclosure herein of any device and/or system corresponds to a disclosure of a method of producing or otherwise providing or otherwise making such. Any disclosure of functionality corresponds to a disclosure of a method action of achieving such.

Any disclosure of a device herein corresponds to a disclosure of using such, including using such to achieve a given functionality. Any disclosure of a method action herein corresponds to a disclosure of a device and/or system for executing such.

Any embodiment or any feature disclosed herein can be combined with any one or more or other embodiments and/or other features disclosed herein, unless explicitly indicated and/or unless the art does not enable such. Any embodiment or any feature disclosed herein can be explicitly excluded from use with any one or more other embodiments and/or other features disclosed herein, unless explicitly indicated that such is combined and/or unless the art does not enable such exclusion.

While various embodiments of the present invention have been described above, it should be understood that they have been presented by way of example only, and not limitation. It will be apparent to persons skilled in the relevant art that various changes in form and detail can be made therein without departing from the spirit and scope of the invention.

What is claimed is:

1. An apparatus, comprising:
an implantable housing;
implantable electronics hermetically sealed within at least a portion of the implantable housing; and
an implantable inductance coil, wherein
an outer diameter of the inductance coil is less than 17.5 mm and located in its entirety within 5 mm of portion(s) of the housing, and
wherein the apparatus is fully implantable within a human.

2. (canceled)

3. The apparatus of claim 1, wherein:
the apparatus is a miniaturized implantable portion of a cochlear implant.

4. The apparatus of claim 1, wherein:
the at least a portion of the housing is implanted in a mastoid bone of a human recipient; and
the coil is located above the electronics with respect to the direction above a surface of the mastoid bone and the coil extends outboard of outer walls of the housing.

5. The apparatus of claim 1, wherein:
the apparatus is configured for functional implantation in a recipient with the housing implanted in a mastoid of a human skull and the coil implanted in the recipient so that the coil is adjacent an ear canal side wall of the recipient.

6. The apparatus of claim 1, wherein:
the apparatus is configured for functional implantation in a recipient with the at least a portion of the housing implanted in the mastoid of a human skull, and the coil implanted in the recipient so that the coil is shadowed by a pinna of the recipient when viewed directly from the side of the human skull.

7. The apparatus of claim 1, wherein:
the apparatus is in receptive wireless signal communication with a behind-the-ear (BTE) device or an in-the-ear (ITE) device worn behind an ear of a human or in an ear canal of a human, respectively; and
the wireless signal communication emanates directly from a respective body of the BTE or ITE device, and in the case of the ITE device, the signal enters the ear canal wall and passes therethrough to reach the apparatus.

8. (canceled)

9. The apparatus of claim 1, wherein:
the at least a portion of the housing has a length, a width and a height, wherein the height is at least half as that of a maximum diameter of the coil, wherein the height is measured in a direction parallel to a longitudinal axis of the coil.

10. A system, comprising:
an implantable component configured to be fully implantable in a human; and
an external component configured to be, in its totality, retained on a head of the human via at least one of behind-the-ear structure or in-the-ear structure, wherein
the external component is configure to be in inductance signal communication with the implantable component.

11. The system of claim 10, wherein:
the system is configured such that the inductance signal communication is maintainable through skin of the human in the absence of magnetic attraction between the implantable component and the eternal component when the system is subject to a 1 G acceleration in any direction tangent to a skin surface at the site of inductance signal communication.

12. The system of claim 10, wherein:
the external component includes a behind-the-ear device including a housing configured to be positioned behind-the ear of the recipient; and
an inductance coil is located in and/or on the housing, which inductance coil establishes the inductance signal communication with the implantable component in conjunction with an implantable coil of the implantable component.

13-14. (canceled)

15. The system of claim 10, wherein:
the implantable component includes an inductance coil that establishes the inductance signal communication with the external component in conjunction with an external coil of the external component; and
the implantable component is configured to be implanted in the recipient such that an outer profile of a pinna of a 50 percentile female of 40 years of age born in the United States of America encompasses the footprint of the coil of the inductance coil when viewed directly from a side of the head of the recipient.

16. The system of claim 10, wherein:
the system is a partially implantable cochlear implant, and the implantable component is the implantable component of the partially implantable cochlear implant.

17. The system of claim 10, wherein:
the system is a partially implantable cochlear implant, and the implantable component is the implantable component of the partially implantable cochlear implant; and
the implantable component has an inductance coil that has a diameter no greater than 17.5 mm.

18. A surgical implantation method, comprising:
accessing a location inside a human; and
surgically implanting an implantable portion of a hearing prosthesis at the location, wherein
the hearing prosthesis is one of a middle ear implant or a cochlear implant powered by transcutaneous inductance power transfer, and
at least one of:
the action of accessing the location inside the human is executed by incising skin with lengths greater than ½ inch only within 3 inches of an inner surface of an ear canal of the human; or
the implantable portion has a height exceeding at least half a maximum width, and the action of accessing the location is executed by drilling and/or excavating into bone only within 2 inches of an inner surface of the ear canal of the human.

19. The method of claim 18, wherein:
the method includes making an endaural incision, wherein the endaural incision extends no more than 2 inches, and wherein the endaural incision is the largest incision made during the surgical method.

20. The method of claim 18, wherein:
the method includes making an endaural incision, wherein the endaural incision is a distinct incision such that during the action of surgically implanting the implantable portion, the distinct incision is no longer than 2 inches.

21. The method of claim 18, wherein:
the action of surgically implanting the implantable portion is executed with the recipient under only local anesthesia and sedation.

22. The method of claim 18, wherein:
wherein from the time at the beginning of commencement of making the first incision to the point where the first incision can be closed, a time that has elapsed is no more than 45 minutes.

23. The method of claim 18, wherein:
the surgical implantation method is executed such that upon closure, all implanted components are within 2 inches of a skin surface of the ear canal.

24. (canceled)

25. The method of claim 18, wherein:
the action of accessing a location inside the human is executed by creating a suprameatal well or a suprameatal bore; and
the action of surgically implanting the implantable portion of the hearing prostheses includes placing a receiver-stimulator the hearing prostheses at least partially within the well or bore.

26-28. (canceled)
